(19) United States
(12) Patent Application Publication
(10) Pub. No.: US 2024/0319192 A1
(43) Pub. Date: Sep. 26, 2024

(54) USE OF IMMUNE CONTENT SCORES DIAGNOSTICALLY TO PREDICT RESPONSIVENESS OF PROSTATE CANCER PATIENTS TO IMMUNOTHERAPY

(71) Applicants: Decipher Biosciences, Inc., San Diego, CA (US); Northwestern University, Evanston, IL (US)

(72) Inventors: Elai Davicioni, San Diego, CA (US); Yang Liu, Vancouver (CA); Edward Matthew Schaeffer, Winnetka, IL (US); Adam Benjamin Weiner, Chicago, IL (US)

(21) Appl. No.: 18/261,638

(22) PCT Filed: Jan. 13, 2022
(86) PCT No.: PCT/US2022/012371

§ 371 Date: Jul. 14, 2023

Related U.S. Application Data
(60) Provisional application No. 63/138,170, filed on Jan. 15, 2021.

(51) Int. Cl. G01N 33/574; C12Q 1/6851; C12Q 1/6886; G01N 33/569

(52) U.S. Cl. CPC G01N 33/57434; C12Q 1/6851; C12Q 1/6886; G01N 33/56966; C12Q 2600/106; C12Q 2600/154; C12Q 2600/158

(57) ABSTRACT

The present disclosure pertains to the field of personalized medicine and methods for treating prostate cancer. In particular, the disclosure relates to the use of immune content scores to identify individuals in need of treatment for prostate cancer who are likely to be responsive to immunotherapy. The disclosure further relates to methods and immune content scores that identify tumors with enhanced immune activity and are prognostic for metastasis-free and disease-free survival for cancer patients.

### RELATED APPLICATIONS

This application claims priority to the U.S. Provisional Patent Application Ser. No. 63/138,170, filed on Jan. 15, 2021, which is hereby incorporated by reference herein in its entirety.

### FIELD

The present disclosure pertains to the field of personalized medicine and methods for treating prostate cancer. In particular, the disclosure relates to the use of immune content scores to identify individuals in need of treatment for prostate cancer who are likely to be responsive to immunotherapy. The disclosure further relates to methods and immune content scores that identify tumors with enhanced immune activity and are prognostic for metastasis-free and disease-free survival for cancer patients.

### BACKGROUND

Cancer is the uncontrolled growth of abnormal cells anywhere in a body. The abnormal cells are termed cancer cells, malignant cells, or tumor cells. Many cancers and the abnormal cells that compose the cancer tissue are further identified by the name of the tissue that the abnormal cells originated from (for example, prostate cancer). Cancer cells can proliferate uncontrollably and form a mass of cancer cells. Cancer cells can break away from this original mass of cells, travel through the blood and lymph systems, and lodge in other organs where they can again repeat the uncontrolled growth cycle. This process of cancer cells leaving an area and growing in another body area is often termed metastatic spread or metastatic disease. For example, if prostate cancer cells spread to a bone (or anywhere else), it can mean that the individual has metastatic prostate cancer.

Standard clinical parameters such as tumor size, grade, lymph node involvement and tumor-node-metastasis (TNM) staging (American Joint Committee on Cancer) may correlate with outcome and serve to stratify patients with respect to (neo)adjuvant chemotherapy, immunotherapy, antibody therapy and/or radiotherapy regimens. Incorporation of molecular markers in clinical practice may define tumor subtypes that are more likely to respond to targeted therapy. However, stage-matched tumors grouped by histological or molecular subtypes may respond differently to the same treatment regimen. Additional key genetic and epigenetic alterations may exist with important etiological contributions. A more detailed understanding of the molecular mechanisms and regulatory pathways at work in cancer cells and the tumor microenvironment (TME) could dramatically improve the design of novel anti-tumor drugs and inform the selection of optimal therapeutic strategies. The development and implementation of diagnostic, prognostic and therapeutic biomarkers to characterize the biology of each tumor may assist clinicians in making important decisions with regard to individual patient care and treatment.

This background information is provided for the purpose of making known information believed by the applicant to be of possible relevance to the present disclosure. No admission is necessarily intended, nor should be construed, that any of the preceding information constitutes prior art against the present disclosure.

### SUMMARY

Embodiments of the disclosure are based on the discovery of immune content scores that are useful for identifying individuals in need of treatment for prostate cancer who will benefit from immunotherapy. The disclosure provides immune content scores that are useful for identifying prostate tumors with enhanced immune activity. The disclosure provides immune content scores that are useful for identifying cancer patients that are likely to respond to immunotherapy. The disclosure provides immune content scores that are useful for identifying immunoresponsive tumors. The disclosure provides immune content scores that are prognostic for metastasis-free and/or disease-free survival in prostate cancer patients. The disclosure provides methods of treatment comprising utilizing immune content scores that are to identify a subject having a prostate tumor with enhanced immune activity, and treating the identified subject with an immunotherapy.

In some embodiments, the disclosure provides a method for predicting benefit from immunotherapy for a subject who has prostate cancer, the method comprising: assaying a level of immune content in a biological sample comprising immune cells and/or prostate cancer cells from the subject, wherein an abnormal level of immune content indicates that the subject is likely to be responsive to immunotherapy. In some embodiments, the immune content is plasma cells, tertiary lymphoid structure activity, IgG expression, interferon gamma (IFNG) signaling, and/or natural killer (NK) cell activity. In some embodiments, the plasma cell levels are increased in the sample. In some embodiments, the tertiary lymphoid structure activity level is increased in the sample. In some embodiments, the IgG expression level is increased in the sample. In some embodiments, the IFNG signaling level is increased in the sample. In some embodiments, the NK cell activity level is increased in the sample. In some embodiments, the CD138/syndecan-1 level is increased in the sample. In some embodiments, the expression level for any one or more of the following markers KLK3, KLK2, FKBP5, STEAP1, STEAP2, PPAP2A, RAB3B, ACSL3, and NKX3-1 is altered in the sample. In some embodiments, the expression level for any one of the following markers CD27, HLA-F, HLA-B, HLA-A, B2M, HLA-E, Act CD4, Act CD8, Tem CD8, TAP2, HLA-DPB1, ICOS, TAP-1, HLA-C, HLA-DPA1 is increased in the sample. In some embodiments, the expression level for any one of the following markers MDSC, ID01, CTLA-4, Treg, PD-L2, TIM3, PD-L1, TIGIT, PD-1, LAG3 compared to a control sample is decreased in the sample. In some embodiments, the method is performed prior to treatment of the patient with immunotherapy. In some embodiments, the immune content is correlated with prolonged recurrence-free survival following surgery for prostate cancer. In some embodiments, the patient is undergoing immunotherapy. In some embodiments, the prostate cancer is biochemically recurrent prostate cancer. In some embodiments, the method is performed after the patient undergoes radical prostatectomy. In some embodiments, the method further comprises performing a radical prostatectomy on the patient. In some embodiments, the prostate cancer is metastatic prostate cancer. In some embodiments, the prostate cancer is metastatic castrate-resistant prostate cancer. In some embodiments, the biological sample is a biopsy. In some embodiments, the biological sample is a tumor sample. In some embodiments, the subject is black or of African descent. In some embodiments, the disclosure provides a method comprising: obtaining a biological sample from a subject having prostate cancer, wherein the sample comprises nucleic acids; and detecting the level of expression of one or more targets selected from CCR7, CXCR5, SELL, LAMP3, CCL19, CCL21, and CXCL13. In some embodiments, the disclosure provides a method comprising: obtaining a biological sample from a subject having prostate cancer, wherein the sample comprises nucleic acids; and detecting the level of expression of one or more targets selected from IGLC2, IGKC, IGHG1, IGHA1, IGHM, and IGHG3. In some embodiments, the disclosure provides a method comprising: obtaining a biological sample from a subject having prostate cancer, wherein the sample comprises nucleic acids; and detecting the level of expression of one or more targets selected from IL15, XCL1, XCL2, CCL5, FLT3LG, GZMA, GZMB, and FASLG. In some embodiments, the disclosure provides a method comprising: obtaining a biological sample from a subject having prostate cancer, wherein the sample comprises plasma cells; and detecting the level of expression of one or more targets selected from CD138/syndecan-1. In some embodiments, the disclosure provides a method comprising: obtaining a biological sample from a subject having prostate cancer, wherein the sample comprises prostate cancer cells; and detecting the level of expression of one or more targets selected from CCR7, CXCR5, SELL, LAMP3, CCL19, CCL21, and CXCL13. In some embodiments, the disclosure provides a method comprising: obtaining a biological sample from a subject having prostate cancer, wherein the sample comprises nucleic acids; and detecting the level of expression of one or more targets selected from CD27, HLA-F, HLA-B, HLA-A, B2M, HLA-E, Act CD4, Act CD8, Tem CD8, TAP2, HLA-DPB1, ICOS, TAP-1, HLA-C, HLA-DPA1. In some embodiments, the disclosure provides a method comprising: obtaining a biological sample from a subject having prostate cancer, wherein the sample comprises nucleic acids; and detecting the level of expression of one or more targets selected from MDSC, ID01, CTLA-4, Treg, PD-L2, TIM3, PD-L1, TIGIT, PD-1, LAG3. In some embodiments, the disclosure provides a method comprising: obtaining a biological sample from a subject having prostate cancer, wherein the sample comprises nucleic acids; and detecting the level of expression of GZMA and PRF1. In some embodiments the sample comprises immune cells. In some embodiments that sample contains prostate cancer cells. In some embodiments the sample comprises immune and prostate cancer cells.

In some embodiments, the disclosure provides a method for treating a patient for prostate cancer, the method comprising: determining whether or not the patient is likely to be responsive to immunotherapy according to the method of claim 1; and administering immunotherapy to the patient if the patient is identified as likely to be responsive to immunotherapy, or administering a cancer treatment other than immunotherapy to the patient if the patient is not identified as likely to be responsive to immunotherapy. In some embodiments, the immunotherapy comprises cellular immunotherapy, antibody immunotherapy, or cytokine immunotherapy. In some embodiments, the immunotherapy is sipuleucel-T. In some embodiments, the methods further comprise performing surgery, radiation therapy, chemotherapy, hormonal therapy, biologic therapy, or any combination thereof. In some embodiments the sample comprises immune cells. In some embodiments that sample contains prostate cancer cells. In some embodiments the sample comprises immune and prostate cancer cells.

In some embodiments, the disclosure provides a method for determining a treatment for a patient who has prostate cancer, the method comprising: determining whether or not the patient is likely to be responsive to immunotherapy according to the method of claim 1; and prescribing immunotherapy to the patient if the patient is identified as likely to be responsive to immunotherapy, or prescribing a cancer treatment other than immunotherapy to the patient if the patient is not identified as likely to be responsive to immunotherapy. In some embodiments the sample comprises immune cells. In some embodiments that sample contains prostate cancer cells. In some embodiments the sample comprises immune and prostate cancer cells.

In some embodiments, the disclosure provides a kit for predicting response of a subject to immunotherapy, the kit comprising agents for measuring levels of immune content in a biological sample comprising immune cells and/or prostate cancer cells. In some embodiments, the immune content is plasma cells, tertiary lymphoid structure activity, IgG expression, interferon gamma (IFNG) signaling, and/or natural killer (NK) cell activity. In some embodiments, the kit comprises agents for measuring the levels of plasma cells, tertiary lymphoid structure activity, IgG expression, interferon gamma (IFNG) signaling, and/or natural killer (NK) cell activity. In some embodiments, the kit further comprises information, in electronic or paper form, comprising instructions on how to determine if a subject is likely to be responsive to immunotherapy. In some embodiments, the kit further comprises one or more control reference samples. In some embodiments the sample comprises immune cells. In some embodiments that sample contains prostate cancer cells. In some embodiments the sample comprises immune and prostate cancer cells.

In some embodiments, the disclosure provides a method of diagnosing, prognosing, determining the progression of cancer, or predicting benefit from immunotherapy in a subject with prostate cancer, comprising: assaying a level of immune content in a biological sample comprising immune cells and/or prostate cancer cells from the subject; and diagnosing, prognosing, determining the progression of cancer, or predicting benefit from immunotherapy in the subject based on the level of immune content in the biological sample. In some embodiments, a higher level of immune content compared to a control reference value indicates that the patient will likely respond to immunotherapy. In some embodiments, the immune content is plasma cells, tertiary lymphoid structure activity, IgG expression, interferon gamma (IFNG) signaling, and/or natural killer (NK) cell activity. In some embodiments, the plasma cell levels are increased in the sample. In some embodiments, the tertiary lymphoid structure activity level is increased in the sample. In some embodiments, the IgG expression level is increased in the sample. In some embodiments, the IFNG signaling level is increased in the sample. In some embodiments, the NK cell activity level is increased in the sample. In some embodiments, the CD138/syndecan-1 level is increased in the sample. In some embodiments, the expression level for any one or more of the following markers KLK3, KLK2, FKBP5, STEAP1, STEAP2, PPAP2A, RAB3B, ACSL3, and NKX3-1 is altered in the sample. In some embodiments, the expression level for any one of the following markers CD27, HLA-F, HLA-B, HLA-A, B2M, HLA-E, Act CD4, Act CD8, Tem CD8, TAP2, HLA-DPB1, ICOS, TAP-1, HLA-C, HLA-DPA1 is increased in the sample. In some embodiments, the expression level for any one of the following markers MDSC, ID01, CTLA-4, Treg, PD-L2, TIM3, PD-L1, TIGIT, PD-1, LAG3 compared to a control sample is decreased in the sample. In some embodiments, the method is performed prior to treatment of the patient with immunotherapy. In some embodiments, the immune content is correlated with prolonged recurrence-free survival following surgery for prostate cancer. In some embodiments, the patient is undergoing immunotherapy. In some embodiments, the prostate cancer is biochemically recurrent prostate cancer. In some embodiments, the method is performed after the patient undergoes radical prostatectomy. In some embodiments, the methods of the disclosure further comprise performing a radical prostatectomy on the patient. In some embodiments, the prostate cancer is metastatic prostate cancer. In some embodiments, the prostate cancer is metastatic castrate-resistant prostate cancer. In some embodiments, the biological sample is a biopsy. In some embodiments, the biological sample is a tumor sample. In some embodiments, the subject is black or of African descent. In some embodiments, the immunotherapy is cellular immunotherapy, antibody immunotherapy, and/or cytokine immunotherapy. In some embodiments, the immunotherapy is sipuleucel-T. In some embodiments the sample comprises immune cells. In some embodiments that sample contains prostate cancer cells. In some embodiments the sample comprises immune and prostate cancer cells.

These and some embodiments of the subject disclosure will readily occur to those of skill in the art in view of the disclosure herein.

### INCORPORATION BY REFERENCE

All publications, patents, and patent applications mentioned in this specification are herein incorporated by reference for the disclosures referenced herein and in their entireties to the same extent as if each individual publication, patent, or patent application was specifically and individually indicated to be incorporated by reference.

### BRIEF DESCRIPTION OF THE DRAWINGS

FIGS. 1A-1K set forth data showing prostate tumors from black men have increased quantities of plasma cells which is associated with increased immune activity. In JHMI, tumors with lower lymphocyte evasion scores were enriched with PC-B (FIG. 1A) suggesting PC-B is more susceptible to lymphocyte infiltration (p-value from Cochrane-Armitage test for trend). Accordingly, we note overall tumor immune content was higher in PC-B (FIG. 1B). The immune content was deconvoluted and applied to our estimate of immune content represented by the different colorings of each bar in (FIG. 1C). Within JHMI, the quantity of several cell types differed by race with increased plasma cells in PC-B representing the largest difference (Wilcoxon Rank Sum: *=FDR p<0.05; ***=FDR p<0.001; FIG. 1D). Of these, increased plasma cell content in PC-B was the only difference in both validation cohorts (FIG. 1E), all p-values based on Wilcoxon Rank Sum). Gene signatures of chronic and acute inflammation and IFNG were elevated in PC-B in JHMI (**p<0.01, ***p<0.001; FIG. 1F). From linear regressions, we demonstrate increasing quartiles of plasma cells were associated with greater expression of inflammation and IFNG but not for CD8+ T-cells (FIG. 1G; *p<0.05, **p<0.01, ***p<0.001). Antigen presentation, cytokine-mediated signaling, and release of tumor-specific antibodies are increased when B-cells localize to tertiary lymphoid structures. We show tumors with high tertiary lymphoid structure activity and plasma cells are enriched with high inflammation, IFNG, IgG expression, and Black race (FIGS. 1H-1K). Dashed horizontal and vertical lines indicate the median for tertiary lymphoid structure activity and plasma cells, respectively. Abbreviations: JHMI, Johns Hopkins Medical Institute; TCGA, The Cancer Genome atlas; DVA, Durham Veterans Affairs; PC-B, prostate cancer from Black men; PC-W, prostate cancer from White men; IFNG, interferon gamma.

FIGS. 2A-2J set forth data showing tumors with high plasma cell content define a subset of prostate cancer improved oncologic outcomes and NK activity. Increasing plasma cell content is associated with longer disease-free survival while increasing CD8+ T-cell content is not (FIG. 2A) even after adjusting for clinical covariates in Cox regressions FIGS. 2B-2C). In TCGA, plasma cell content and IgG expression were both higher in tumors with any IgG3 to 1 subclass switch recombination, a measure of B-cell clonality (FIG. 2D; Wilcoxon Rank Sum: *=p<0.05; ***=p<0.001). We also show while high T-cell activity as represented by cytolytic activity was not associated with metastasis-free survival in JHMI, high IgG expression was associated with longer survival duration (FIG. 2E) even after adjusting for clinical covariates in Cox regression (FIG. 2F). Because plasma cells and IgG contribute to antibody-dependent cellular cytotoxicity in terms of tumor immunogenicity which is classically defined by NK cell activity in response to IgG, we noted NK activity is elevated in tumors with high levels of plasma cells and IgG expression (FIG. 2G; Wilcoxon Rank Sum: ***=p<0.001). Within DVA, increasing plasma cells and IgG expression showed a continuous relationship between these scores and NK activity (FIG. 2H; p-values are from Kruskal-Wallis analyses for trend). Higher NK activity trended towards an association with longer time to metastasis (FIG. 2I) which reached significance after adjusting for clinical covariates (FIG. 2J). All survival estimations were performed using the Kaplan-Meier method and p-values associated with survival curves are all log-rank tests. * next to p-values from Cox regressions denote statistical significance with p<0.05. Abbreviations: JHMI, Johns Hopkins Medical Institute; TCGA, The Cancer Genome atlas; DVA, Durham Veterans Affairs; HR, Hazard ratio; PSA, prostate-specific antigen.

FIGS. 3A-3O set forth data showing a second method for immune cell deconvolution, Microenvironment Cell Populations-counter (MCP-counter), was applied to confirm findings from MySort (FIG. 3A; p-values based on Wilcoxon Rank Sum). Using a DNA-methylation-based signatures in TCGA and a grade-matched cohort of 135 PC-B and 135 PC-W B-cell (CD19) lineage signals were again higher in PC-B tumors (FIGS. 3B-3C; p-values based on Wilcoxon Rank Sum). CD79a immunostaining in a tumor from a Black patient shows abundant positive cells (brown; FIG. 3D, top panel). Automated digital image cell segmentation identifies the density of positive cells per mm² of tissue sampled (red circles; FIG. 3D, bottom panel). Images of CD138 immunostaining in prostate cancer tissue microarray cores show cases with high levels of CD138+ plasma cells (arrows; FIG. 3E). Manual quantification was required due to patchy CD138 immunopositivity in background benign epithelial cells (arrowhead). All images reduced from 200× magnification. CD79a+ and CD138+ densities were higher in PC-B (FIGS. 3F-3G; p-values based on Wilcoxon Rank Sum). Similar to results from JHMI (FIG. 3F), trends toward greater levels of inflammation and IFNG were noted in tumors from Black in TCGA and DVA (FIG. 3H; p-values based on Wilcoxon Rank Sum). Because plasma cell IgG production is augmented in the presence of IFNG expression and tertiary lymphoid structures (TLS), and both IFNG expression, TLS signatures, and plasma cells are present to greater extents in PC-B, we sought to derive a measure of IgG expression. Hierarchical clustering was performed in JHMI using an IgG gene set originally designed for breast cancer. Six genes in this gene set tended to cluster within PC tumors from JHMI (FIG. 3I) and were ultimately included in our IgG signature. From the geometric mean expression of these genes, we created an IgG expression signature which correlated well with plasma cell content (FIG. 3J; Spearman's correlation coefficient r=0.65, p<0.001). One tumor from the highest and lowest pentile of TLS signature scores were randomly selected for immunostaining (FIG. 3K). At low magnification, discrete lymphoid aggregates can be seen in the tumor with the high TLS score surrounding the areas of tissue microarray tumor punch samplings. The box in the upper panel of the high TLS score tumor represents the area magnified in the left most tumor shown in (FIG. 3L). Three randomly selected tumors in the highest pentile of TLS scores underwent immunostaining for CD20, CD3, and CD138 on adjacent slides (FIG. 3L; top panels which were then merged and pseudocolored (FIG. 3L; lower panels with same orientation as top panels) to demonstrate discrete lymphoid aggregates of T- and B-cells, with scattered adjacent CD138+ cells consistent with the presence of TLS. Holes in the tissue represent areas that were punched for RNA isolation and transcriptomic analyses (FIGS. 3K-3L). An assessment of plasma cell content based on race was underpowered in a cohort of men with metastatic castrate resistant prostate cancer (FIG. 3M; p-value based on Wilcoxon Rank Sum). PC-B is less likely to be defined by ERG gene fusion, thus we sought to determine if the association between increased plasma cell content and Black race was related to their propensity to be ERG negative. In both JHMI (FIG. 3N) and DVA (FIG. 3O), from linear regressions adjusting for race and ERG status, the interaction terms between Black race and ERG+ status were not statistically significant suggesting Black race is associated with increased plasma cell content independent of ERG status. Abbreviations: JHMI, Johns Hopkins Medical Institute; TCGA, The Cancer Genome atlas; DVA, Durham Veterans Affairs; PC-B, Prostate cancer from Black men; PC-W, prostate cancer from White men; IFNG, interferon gamma, TLS, tertiary lymphoid structure, OR, odds ratio.

FIGS. 4A-4J set forth data showing in JHMI, there was no association between grade group and plasma cell content and IgG expression (FIG. 4A) while in TCGA higher grade tumors tended to have higher plasma cell content (FIG. 4B; both p-values from Kruskal-Wallis test). Similar to JHMI, in TCGA after adjusting for clinical covariates in Cox regressions, higher quantities of plasma cells but not CD8+ T-cells trended towards longer disease-free survival (FIGS. 4C-4D). A higher proportion of PC-B were categorized as having any IgG3 to 1 subclass switch recombination on 361 of 468 patients in our cohort from TCGA although this was not statistically significant (FIG. 4E). Using data from The Cancer Immune Atlas project (https://tcia.at/home) on the neoantigen burden for 420 of 468 patients in our cohort from TCGA and fraction genome altered as defined as the length of segments with log 2 or linear copy number alteration value larger than 0.2 divided by the length of all segments measured as available at https://www.cbioportal.org/, we show both neoantigen burden and fraction genome altered correlated poorly with plasma cells, IgG, and NK activity (FIGS. 4F-4G; shown are Spearman correlation coefficients). Similarly, there was no association between neoantigen burden or fraction genome altered and race (FIGS. 4H-4I; p-values based on Wilcoxon Rank Sum). The expression level of inflammatory cytokines commonly associated with IgA class switch were not correlated with plasma cell content in JHMI (FIG. 4J; shown are Spearman correlation coefficients). Abbreviations: JHMI, Johns Hopkins Medical Institute; TCGA, The Cancer Genome atlas; HR, hazard ratio; PSA, prostate-specific antigen. IgG3-1, IgG 3 to 1 class-switch recombination. * next to p-values from Cox regressions denote statistical significance with p<0.05.

FIG. 5 sets forth data showing immunohistochemistry validation of the plasma cell signature. In primary prostate tumors from the JHMI cohort with immunohistochemistry data (n=113), plasma cell content based on an expression signature was the only immune cell type that correlated significantly with CD138+ density (Shading is 95% confidence level interval for predictions from a linear model). Abbreviations: JHMI, Johns Hopkins Medical Institute; FDR, false discovery rate.

FIGS. 6A-6C set forth data showing plasma cell content in mCRPC. In bone biopsy specimens from the Stand Up to Cancer 2019 cohort, tumors without prior exposure to taxane chemotherapy or ARSi had increased plasma cell content (a; Wilcoxon Rank Sum; center line, median; box limits, upper and lower quartiles; whiskers, 1.5× interquartile range). In previous studies of mCRPC, higher intratumoral CD38 expression was associated with increased immune suppression. However, in primary tumors CD38 expression did not correlate with quantified CD138+ density on immunohistochemistry or plasma cell content based on the expression signature (b; Shading is 95% confidence level interval for predictions from a linear model). Abbreviations: mCRPC, metastatic castration-resistant prostate cancer; ARSi, androgen receptor signaling inhibitor; JHMI, Johns Hopkins Medical Institute.

FIGS. 7A-7B set forth data showing expression-based biomarkers in high grade localized prostate cancer based on plasma cell content. Within the Genomic Resource Information Database (GRID) cohort (high grade primary prostate cancer; n=785), tumors with high plasma cell content were defined as content greater than the mean expression+1 standard deviation (a). Despite having similar genomic risk scores, tumors with high plasma cell content were predicted to have more favorable responses to immunotherapies and RT and lower AR-activity suggesting greater resistance to androgen deprivation therapy (all Wilcoxon Rank Sum; center line, median; box limits, upper and lower quartiles; whiskers, 1.5× interquartile range). Abbreviations: AR, androgen receptor; RT, radiation therapy.

FIGS. 8A-8B set forth data showing immune pathways in high grade localized prostate cancer based on plasma cell content. Within the Genomic Resource Information Database (GRID) cohort (high grade primary prostate cancer; n=785), tumors with high plasma cell content expressed increased pathways in immune activation (a; Wilcoxon Rank Sum FDR P-values; above dashed line represents FDR P<0.05) resulting in immunophenoscores suggestive of increased susceptibilities to immunotherapies (b; Wilcoxon Rank Sum; center line, median; box limits, upper and lower quartiles; whiskers, 1.5× interquartile range). Abbreviation: FDR, false discovery rate.

FIGS. 9A-9C set forth data showing master regulators in primary prostate cancer with high plasma cell content. Within the GRID cohort (n=784), MR analysis revealed transcription factors with increased and decreased activity in tumors with high plasma cell content (a). There was minimal heterogeneity in the plasma cell-high tumors related to differentially activated MRs (b). Multivariable Cox proportional hazards regressions within the Johns Hopkins Medical Institute cohort (n=498), adjusting for serum prostate-specific antigen, tumors stage, and tumors grade (see Methods), showed the three MRs overactivated in plasma cell-low tumors were associated with shorter time to metastatic recurrence following radical prostatectomy (c). Abbreviations: MR, master regulator; HR, hazard ratio; CI, confidence interval; GRID, Genomic Resource Information Database.

### DETAILED DESCRIPTION

The practice of the present disclosure will employ, unless otherwise indicated, conventional methods of medicine, biochemistry, molecular biology and recombinant DNA techniques, within the skill of the art. Such techniques are explained fully in the literature. See, e.g., Immunotherapy of Cancer (Advances in Cancer Research Volume 128, X. Wang and P. B. Fisher eds., Academic Press, 2015); Prostate Cancer: Science and Clinical Practice (J. H. Mydlo and C. J. Godec eds., Academic Press, 2^{nd} edition, 2015); Prostate Cancer: Biochemistry, Molecular Biology and Genetics (Protein Reviews 16, D. J. Tindall ed., Springer, 2013); A. L. Lehninger, Biochemistry (Worth Publishers, Inc., current addition); Sambrook, et al., Molecular Cloning: A Laboratory Manual (3^{rd} Edition, 2001); and Methods In Enzymology (S. Colowick and N. Kaplan eds., Academic Press, Inc.).

Before describing the present disclosure in detail, it is to be understood that this disclosure is not limited to particular formulations or process parameters as such may, of course, vary. It is also to be understood that the terminology used herein is for the purpose of describing particular embodiments of the disclosure only, and is not intended to be limiting.

Although a number of methods and materials similar or equivalent to those described herein can be used in the practice of the present disclosure, embodiments of materials and methods are described herein.

Embodiments of the present disclosure are based on the discovery of immune content scores that can be used to identify individuals likely to respond to immunotherapy who are in need of treatment for prostate cancer.

In order to further an understanding of the disclosure, a more detailed discussion is provided below regarding the identified immune content scores and methods of screening and treating subjects for prostate cancer.

### Immune Content Scores

Immune content scores can be utilized to identify prostate cancer patients that will likely benefit from immunotherapy. Such immune content scores can be utilized to detect increased levels of plasma cells, tertiary lymphoid structure activity, immunoglobulin G (IgG) expression, interferon gamma (IFNG) signaling, and/or natural killer (NK) cell activity which indicate that a subject is likely to benefit from immunotherapy. Additionally, immune content scores can be used to identify subjects with low plasma cells, tertiary lymphoid structure activity, immunoglobulin G (IgG) expression, interferon gamma (IFNG) signaling, and/or natural killer (NK) cell activity that may still also potentially benefit from other cancer therapies. In some embodiments, expression signatures are used to measure levels of plasma cells, tertiary lymphoid structure activity, immunoglobulin G (IgG) expression, interferon gamma (IFNG) signaling, and/or natural killer (NK) cell activity.

Exemplary markers for immune content scores include plasma cells, tertiary lymphoid structure activity, immunoglobulin G (IgG) expression, interferon gamma (IFNG) signaling, and/or natural killer (NK) cell activity, respectively. Accordingly, levels of plasma cells, tertiary lymphoid structure activity, immunoglobulin G (IgG) expression, interferon gamma (IFNG) signaling, and/or natural killer (NK) cell activity provide an immune content score.

In one aspect the disclosure includes a method of diagnosing, prognosing, determining the progression of cancer, or predicting benefit from immunotherapy in a subject with prostate cancer, comprising: assaying a level of immune content in a biological sample comprising immune cells and/or prostate cancer cells from the subject; and diagnosing, prognosing, determining the progression of cancer, or predicting benefit from immunotherapy in the subject based on the level of immune content in the biological sample. Additionally, a cancer patient may also be identified as likely be responsive to immunotherapy using an immune content score based on the levels in a biological sample of plasma cells, tertiary lymphoid structure activity, IgG expression, interferon gamma (IFNG) signaling, and/or natural killer (NK) cell activity (see Examples).

Thus, the methods described herein may be used to determine if a patient should be treated for prostate cancer with immunotherapy or some other method. The methods can be performed prior to treatment of a subject with immunotherapy to determine if the subject is likely to benefit from immunotherapy, or can be performed while the subject is undergoing immunotherapy to help evaluate whether continued treatment is likely to be efficacious.

A patient is selected for treatment with immunotherapy if the patient is identified as being likely to be responsive to immunotherapy based on the level of immune content in a biological sample from the patient, as described herein. Immunotherapy may include, but it not limited to, cellular immunotherapy, antibody immunotherapy, or cytokine immunotherapy. The immunotherapy, sipuleucel-T, is specifically envisaged for use in the methods of the disclosure. In addition, patients, especially those not identified as likely to benefit from immunotherapy, may be administered other cancer treatments such as, but not limited to, surgery, radiation therapy, chemotherapy, hormonal therapy, biologic therapy, or any combination thereof.

### Targets and Immune Content Signatures

In some instances, assaying the expression level of a plurality of genes comprises detecting and/or quantifying a plurality of target analytes. In some embodiments, assaying the expression level of a plurality of genes comprises sequencing a plurality of target nucleic acids. In some embodiments, assaying the expression level of a plurality of biomarker genes comprises amplifying a plurality of target nucleic acids. In some embodiments, assaying the expression level of a plurality of biomarker genes comprises conducting a multiplexed reaction on a plurality of target analytes.

The methods disclosed herein often comprise assaying the expression level of a plurality of targets. A tertiary lymphoid structure signature may be used in the methods of the disclosure. In some instances, the plurality of targets is a list of tertiary lymphoid structure (TLS) genes, (Cabrita, R. et al. Nature 577, 561-565 (2020) and Dieu-Nosjean, M.-C., et al. Trends in Immunology 35, 571-580 (2014)). In some instances, the TLS signature reflects the activity and presence of tertiary lymphoid structures within the TME as the geometric mean expression of seven genes (CCR7, CXCR5, SELL, LAMP3, CCL19, CCL21, and CXCL13). In some embodiments, the disclosure provides a method comprising: obtaining a biological sample from a subject having prostate cancer, wherein the sample comprises nucleic acids; and detecting the level of expression of one or more targets selected from CCR7, CXCR5, SELL, LAMP3, CCL19, CCL21, and CXCL13.

ERG status and PTEN loss signatures may be used in the methods of the disclosure. In some instances, the plurality of targets is a validated transcriptome-based signature with 95% accuracy for predicting ERG+/− status and PTEN loss (Tomlins, S. A. et al. European Urology 68, 555-567 (2015)).

Plasma cell levels may be measured with immunohistochemistry. In some instances. CD138/syndecan-1, a marker of plasma cells, is used to measure plasma cell levels. In other instances, quantification of CD138+ cell density is performed, counting all individual cells positive for CD138 in a standardized diameter of tissue core of tumors, normalized by the total mm² of tissue analyzed for each core calculated and taking the mean across the four tumor cores for each patient.

Interferon gamma expression signatures may be used in the methods of the disclosure. In some instances, a Hallmark Gene Set Collection of interferongamma (IFNG; HALLMARK_INTERFERON_GAMMA_RESPONSE) is used (Liberzon, A. et al. Cell Syst. 1, 417-425 (2015)). Inflammation gene expression signatures may also be used in the methods of the disclosure. In some instances, a Hallmark Gene Set Collection, a measure of genes upregulated during chronic and acute inflammation (HALLMARK_INFLAMMATORY_RESPONSE) is used (Liberzon, A. et al. Cell Syst. 1, 417-425 (2015)).

Targets of the disclosure include tumor lymphocytes. In some embodiments, a tumor lymphocyte evasion score is derived and defined by the expression of genes from cell types that are capable of evading lymphocyte infiltration (cancer-associated fibroblasts [CAFs], myeloid-derived suppressor cells [MDSCs], and M2 subtype of tumor-associated macrophages [TAMs]) as described by Jiang et al. (“exclusion” score in previous publication) (Jiang, P. et al. Nat Med 24, 1550-1558 (2018)).

Immunoglobulin G (IgG) expression signatures may be used in the methods of the disclosure. In some instances, an unsupervised hierarchal clustering is applied to a gene-set of proteins related to IgG components and expression (Rody, A. et al. Breast Cancer Research 11, R15 (2009)). Six IgG genes that are highly correlated with each other in prostate cancer tumors are used to determine IgG expression levels (IGLC2, IGKC, IGHG1, IGHA1, IGHM, and IGHG3). In some embodiments, the disclosure provides a method comprising: obtaining a biological sample from a subject having prostate cancer, wherein the sample comprises nucleic acids; and detecting the level of expression of one or more targets selected from IGLC2, IGKC, IGHG1, IGHA1, IGHM, and IGHG3.

Natural Killer (NK) cell activity expression signatures may be used in the methods of the disclosure. In some instances, NK activity is measured using the geometric mean expression of eight genes expressed during NK activation (IL15, XCL1, XCL2, CCL5, FLT3LG, GZMA, GZMB, and FASLG). In some embodiments, the disclosure provides a method comprising: obtaining a biological sample from a subject having prostate cancer, wherein the sample comprises nucleic acids; and detecting the level of expression of one or more targets selected from IL15, XCL1, XCL2, CCL5, FLT3LG, GZMA, GZMB, and FASLG.

T-cell activity expression signatures may be used in the methods of the disclosure. In some instances, cytotoxic T-cell (CD8+) activity is measured as the geometric mean expression of GZMA and PRF1 (Rooney et al. (Cell 160, 48-61 (2015)). In some embodiments, the disclosure provides a method comprising: obtaining a biological sample from a subject having prostate cancer, wherein the sample comprises nucleic acids; and detecting the level of expression of GZMA and PRF1.

### Probes/Primers

The present disclosure provides for a probe set for predicting benefit from immunotherapy and/or diagnosing, monitoring and/or predicting a status or outcome of prostate cancer in a subject comprising a plurality of probes, wherein (i) the probes in the set are capable of detecting an expression level of a plurality of targets; and (ii) the expression level determines the cancer status of the subject with at least about 40% specificity.

The probe set may comprise one or more polynucleotide probes. Individual polynucleotide probes comprise a nucleotide sequence derived from the nucleotide sequence of the target sequences or complementary sequences thereof. The nucleotide sequence of the polynucleotide probe is designed such that it corresponds to, or is complementary to the target sequences. The polynucleotide probe can specifically hybridize under either stringent or lowered stringency hybridization conditions to a region of the target sequences, to the complement thereof, or to a nucleic acid sequence (such as a cDNA) derived therefrom.

The selection of the polynucleotide probe sequences and determination of their uniqueness may be carried out in silico using techniques known in the art, for example, based on a BLASTN search of the polynucleotide sequence in question against gene sequence databases, such as the Human Genome Sequence, UniGene, dbEST or the non-redundant database at NCBI. In some embodiments of the disclosure, the polynucleotide probe is complementary to a region of a target mRNA derived from a target sequence in the probe set. Computer programs can also be employed to select probe sequences that may not cross hybridize or may not hybridize non-specifically.

In some instances, microarray hybridization of RNA, extracted from prostate cancer tissue samples and amplified, may yield a dataset that is then summarized and normalized by the fRMA technique. After removal (or filtration) of cross-hybridizing PSRs, and PSRs containing less than 4 probes, the remaining PSRs can be used in further analysis. Following fRMA and filtration, the data can be decomposed into its principal components and an analysis of variance model is used to determine the extent to which a batch effect remains present in the first 10 principal components.

These remaining PSRs can then be subjected to filtration by a T-test between CR (clinical recurrence) and non-CR samples. Using a p-value cut-off of 0.01, the remaining features (e.g., PSRs) can be further refined. Feature selection can be performed by regularized logistic regression using the elastic-net penalty. The regularized regression may be bootstrapped over 1000 times using all training data; with each iteration of bootstrapping, features that have non-zero co-efficient following 3-fold cross validation can be tabulated. In some instances, features that were selected in at least 25% of the total runs were used for model building.

The polynucleotide probes of the present disclosure may range in length from about 15 nucleotides to the full length of the coding target or non-coding target. In some embodiments of the disclosure, the polynucleotide probes are at least about 15 nucleotides in length. In some embodiments, the polynucleotide probes are at least about 20 nucleotides in length. In some embodiments, the polynucleotide probes are at least about 25 nucleotides in length. In some embodiments, the polynucleotide probes are between about 15 nucleotides and about 500 nucleotides in length. In some embodiments, the polynucleotide probes are between about 15 nucleotides and about 450 nucleotides, about 15 nucleotides and about 400 nucleotides, about 15 nucleotides and about 350 nucleotides, about 15 nucleotides and about 300 nucleotides, about 15 nucleotides and about 250 nucleotides, about 15 nucleotides and about 200 nucleotides in length. In some embodiments, the probes are at least 15 nucleotides in length. In some embodiments, the probes are at least 15 nucleotides in length. In some embodiments, the probes are at least 20 nucleotides, at least 25 nucleotides, at least 50 nucleotides, at least 75 nucleotides, at least 100 nucleotides, at least 125 nucleotides, at least 150 nucleotides, at least 200 nucleotides, at least 225 nucleotides, at least 250 nucleotides, at least 275 nucleotides, at least 300 nucleotides, at least 325 nucleotides, at least 350 nucleotides, at least 375 nucleotides in length.

The polynucleotide probes of a probe set can comprise RNA, DNA, RNA or DNA mimetics, or combinations thereof, and can be single-stranded or double-stranded. Thus the polynucleotide probes can be composed of naturally-occurring nucleobases, sugars and covalent internucleoside (backbone) linkages as well as polynucleotide probes having non-naturally-occurring portions which function similarly. Such modified or substituted polynucleotide probes may provide desirable properties such as, for example, enhanced affinity for a target gene and increased stability.

The system of the present disclosure further provides for primers and primer pairs capable of amplifying target sequences defined by the probe set, or fragments or subsequences or complements thereof. The nucleotide sequences of the probe set may be provided in computer-readable media for in silico applications and as a basis for the design of appropriate primers for amplification of one or more target sequences of the probe set.

Primers based on the nucleotide sequences of target sequences can be designed for use in amplification of the target sequences. For use in amplification reactions such as PCR, a pair of primers can be used. The exact composition of the primer sequences is not critical to the disclosure, but for most applications the primers may hybridize to specific sequences of the probe set under stringent conditions, particularly under conditions of high stringency, as known in the art. The pairs of primers are usually chosen so as to generate an amplification product of at least about 50 nucleotides, more usually at least about 100 nucleotides. Algorithms for the selection of primer sequences are generally known, and are available in commercial software packages. These primers may be used in standard quantitative or qualitative PCR-based assays to assess transcript expression levels of RNAs defined by the probe set. Alternatively, these primers may be used in combination with probes, such as molecular beacons in amplifications using real-time PCR.

A label can optionally be attached to or incorporated into a probe or primer polynucleotide to allow detection and/or quantitation of a target polynucleotide representing the target sequence of interest. The target polynucleotide may be the expressed target sequence RNA itself, a cDNA copy thereof, or an amplification product derived therefrom, and may be the positive or negative strand, so long as it can be specifically detected in the assay being used. Similarly, an antibody may be labeled.

In some multiplex formats, labels used for detecting different targets may be distinguishable. The label can be attached directly (e.g., via covalent linkage) or indirectly, e.g., via a bridging molecule or series of molecules (e.g., a molecule or complex that can bind to an assay component, or via members of a binding pair that can be incorporated into assay components, e.g. biotin-avidin or streptavidin). Many labels are commercially available in activated forms which can readily be used for such conjugation (for example through amine acylation), or labels may be attached through known or determinable conjugation schemes, many of which are known in the art.

Labels useful in the embodiments described herein include any substance which can be detected when bound to or incorporated into the biomolecule of interest. Any effective detection method can be used, including optical, spectroscopic, electrical, piezoelectrical, magnetic, Raman scattering, surface plasmon resonance, colorimetric, calorimetric, etc. A label is typically selected from a chromophore, a lumiphore, a fluorophore, one member of a quenching system, a chromogen, a hapten, an antigen, a magnetic particle, a material exhibiting nonlinear optics, a semiconductor nanocrystal, a metal nanoparticle, an enzyme, an antibody or binding portion or equivalent thereof, an aptamer, and one member of a binding pair, and combinations thereof. Quenching schemes may be used, wherein a quencher and a fluorophore as members of a quenching pair may be used on a probe, such that a change in optical parameters occurs upon binding to the target introduce or quench the signal from the fluorophore. One example of such a system is a molecular beacon. Suitable quencher/fluorophore systems are known in the art. The label may be bound through a variety of intermediate linkages. For example, a polynucleotide may comprise a biotin-binding species, and an optically detectable label may be conjugated to biotin and then bound to the labeled polynucleotide. Similarly, a polynucleotide sensor may comprise an immunological species such as an antibody or fragment, and a secondary antibody containing an optically detectable label may be added.

Chromophores useful in the methods described herein include any substance which can absorb energy and emit light. For multiplexed assays, a plurality of different signaling chromophores can be used with detectably different emission spectra. The chromophore can be a lumophore or a fluorophore. Typical fluorophores include fluorescent dyes, semiconductor nanocrystals, lanthanide chelates, polynucleotide-specific dyes and green fluorescent protein.

In some embodiments, polynucleotides of the disclosure comprise at least 20 consecutive bases of the nucleic acid sequence of a target or a complement thereto. The polynucleotides may comprise at least 21, 22, 23, 24, 25, 27, 30, 32, 35, 40, 45, 50, or more consecutive bases of the nucleic acids sequence of a target as applicable.

The polynucleotides may be provided in a variety of formats, including as solids, in solution, or in an array. The polynucleotides may optionally comprise one or more labels, which may be chemically and/or enzymatically incorporated into the polynucleotide.

In some embodiments, one or more polynucleotides provided herein can be provided on a substrate. The substrate can comprise a wide range of material, either biological, nonbiological, organic, inorganic, or a combination of any of these. For example, the substrate may be a polymerized Langmuir Blodgett film, functionalized glass, Si, Ge, GaAs, GaP, SiO₂, SiN₄, modified silicon, or any one of a wide variety of gels or polymers such as (poly)tetrafluoroethylene, (poly)vinylidenedifluoride, polystyrene, cross-linked polystyrene, polyacrylic, polylactic acid, polyglycolic acid, poly(lactide coglycolide), polyanhydrides, poly(methyl methacrylate), poly(ethylene-co-vinyl acetate), polysiloxanes, polymeric silica, latexes, dextran polymers, epoxies, polycarbonates, or combinations thereof. Conducting polymers and photoconductive materials can be used.

The substrate can take the form of an array, a photodiode, an optoelectronic sensor such as an optoelectronic semiconductor chip or optoelectronic thin-film semiconductor, or a biochip. The location(s) of probe(s) on the substrate can be addressable; this can be done in highly dense formats, and the location(s) can be microaddressable or nanoaddressable.

### Diagnostic Samples

A biological sample containing immune and/or prostate cancer cells is collected from a subject in need of treatment for prostate cancer to evaluate whether a patient will benefit from immunotherapy. Diagnostic samples for use with the systems and in the methods of the present disclosure comprise immune and/or cancer cells suitable for providing immune content information. In principle, the biological sample from which the immune content score is obtained and analyzed can be any material suspected of comprising immune and/or cancerous tissue or cells. The diagnostic sample can be a biological sample used directly in a method of the disclosure. Alternatively, the diagnostic sample can be a sample prepared from a biological sample. In some embodiments more than one sample is obtained from the subject. In some embodiments a sample comprises immune cells but not prostate cancer cells. In some embodiments a sample comprises prostate cancer cells but not immune cells. In some embodiments a sample comprises both immune cells and prostate cancer cells.

In some embodiments, the sample or portion of the sample comprising or suspected of comprising immune and/or cancerous tissue or cells can be any source of biological material, including cells, tissue or fluid, including bodily fluids. Non-limiting examples of the source of the sample include an aspirate, a needle biopsy, a cytology pellet, a bulk tissue preparation or a section thereof obtained for example by surgery or autopsy, lymph fluid, blood, plasma, serum, tumors, and organs. In some embodiments, the sample is from urine. Alternatively, the sample is from blood, plasma or serum. In some embodiments, the sample is from saliva.

The samples may be archival samples, having a known and documented medical outcome, or may be samples from current patients whose ultimate medical outcome is not yet known.

In some embodiments, the sample may be dissected prior to molecular analysis. The sample may be prepared via macrodissection of a bulk tumor specimen or portion thereof, or may be treated via microdissection, for example via Laser Capture Microdissection (LCM).

The sample may initially be provided in a variety of states, as fresh tissue, fresh frozen tissue, fine needle aspirates, and may be fixed or unfixed. Frequently, medical laboratories routinely prepare medical samples in a fixed state, which facilitates tissue storage. A variety of fixatives can be used to fix tissue to stabilize the morphology of cells, and may be used alone or in combination with other agents. Exemplary fixatives include crosslinking agents, alcohols, acetone. Bouin's solution, Zenker solution, Helv solution, osmic acid solution and Carnoy solution.

Crosslinking fixatives can comprise any agent suitable for forming two or more covalent bonds, for example an aldehyde. Sources of aldehydes typically used for fixation include formaldehyde, paraformaldehyde, glutaraldehyde or formalin. Preferably, the crosslinking agent comprises formaldehyde, which may be included in its native form or in the form of paraformaldehyde or formalin. One of skill in the art would appreciate that for samples in which crosslinking fixatives have been used special preparatory steps may be necessary including for example heating steps and proteinase-k digestion; see methods.

One or more alcohols may be used to fix tissue, alone or in combination with other fixatives. Exemplary alcohols used for fixation include methanol, ethanol and isopropanol.

Formalin fixation is frequently used in medical laboratories. Formalin comprises both an alcohol, typically methanol, and formaldehyde, both of which can act to fix a biological sample.

Whether fixed or unfixed, the biological sample may optionally be embedded in an embedding medium. Exemplary embedding media used in histology including paraffin, Tissue-Tek® V.I.P.™, Paramat, Paramat Extra, Paraplast, Paraplast X-tra, Paraplast Plus, Peel Away Paraffin Embedding Wax, Polyester Wax, Carbowax Polyethylene Glycol, Polyfin™, Tissue Freezing Medium TFMFM, Cryo-Gef™, and OCT Compound (Electron Microscopy Sciences, Hatfield, PA). Prior to molecular analysis, the embedding material may be removed via any suitable techniques, as known in the art. For example, where the sample is embedded in wax, the embedding material may be removed by extraction with organic solvent(s), for example xylenes. Kits are commercially available for removing embedding media from tissues. Samples or sections thereof may be subjected to further processing steps as needed, for example serial hydration or dehydration steps.

In some embodiments, the sample is a fixed, wax-embedded biological sample. Frequently, samples from medical laboratories are provided as fixed, wax-embedded samples, most commonly as formalin-fixed, paraffin embedded (FFPE) tissues.

Whatever the source of the biological sample, the target polynucleotide that is ultimately assayed can be prepared synthetically (in the case of control sequences), but typically is purified from the biological source and subjected to one or more preparative steps. The RNA may be purified to remove or diminish one or more undesired components from the biological sample or to concentrate it. Conversely, where the RNA is too concentrated for the particular assay, it may be diluted.

### RNA Extraction

RNA can be extracted and purified from biological samples using any suitable technique. A number of techniques are known in the art, and several are commercially available (e.g., FormaPure nucleic acid extraction kit, Agencourt Biosciences, Beverly MA, High Pure FFPE RNA Micro Kit, Roche Applied Science, Indianapolis, IN). RNA can be extracted from frozen tissue sections using TRIzol (Invitrogen, Carlsbad, CA) and purified using RNeasy Protect kit (Qiagen, Valencia, CA). RNA can be further purified using DNAse I treatment (Ambion, Austin, TX) to eliminate any contaminating DNA. RNA concentrations can be made using a Nanodrop ND-1000 spectrophotometer (Nanodrop Technologies, Rockland, DE). RNA can be further purified to eliminate contaminants that interfere with cDNA synthesis by cold sodium acetate precipitation. RNA integrity can be evaluated by running electropherograms, and RNA integrity number (RIN, a correlative measure that indicates intactness of mRNA) can be determined using the RNA 6000 PicoAssay for the Bioanalyzer 2100 (Agilent Technologies, Santa Clara, CA).

### Kits

Kits for performing the desired method(s) are also provided, and comprise a container or housing for holding the components of the kit, one or more vessels containing one or more nucleic acid(s), and optionally one or more vessels containing one or more reagents. The reagents include those described in the composition of matter section above, and those reagents useful for performing the methods described, including amplification reagents, and may include one or more probes, primers or primer pairs, enzymes (including polymerases and ligases), intercalating dyes, labeled probes, and labels that can be incorporated into amplification products.

In some embodiments, the kit comprises primers or primer pairs specific for those subsets and combinations of target sequences described herein. The primers or pairs of primers suitable for selectively amplifying the target sequences. The kit may comprise at least two, three, four or five primers or pairs of primers suitable for selectively amplifying one or more targets. The kit may comprise at least 5, 10, 15, 20, 30, 40, 50, 60, 70, 80, 90, 100, 110, or more primers or pairs of primers suitable for selectively amplifying one or more targets.

In some embodiments, the primers or primer pairs of the kit, when used in an amplification reaction, specifically amplify a non-coding target, coding target, exonic, or non-exonic target described herein, a nucleic acid sequence corresponding to a target disclosed herein, an RNA form thereof, or a complement to either thereof. The kit may include a plurality of such primers or primer pairs which can specifically amplify a corresponding plurality of different amplify a non-coding target, coding target, exonic, or non-exonic transcript described herein, a nucleic acid sequence corresponding to a target disclosed herein, RNA forms thereof, or complements thereto. At least two, three, four or five primers or pairs of primers suitable for selectively amplifying the one or more targets can be provided in kit form. In some embodiments, the kit comprises from five to fifty primers or pairs of primers suitable for amplifying the one or more targets.

The reagents may independently be in liquid or solid form. The reagents may be provided in mixtures. Control samples and/or nucleic acids may optionally be provided in the kit. Control samples may include tissue and/or nucleic acids obtained from or representative of tumor samples from patients showing no evidence of disease, as well as tissue and/or nucleic acids obtained from or representative of tumor samples from patients that develop systemic cancer.

The nucleic acids may be provided in an array format, and thus an array or microarray may be included in the kit. The kit optionally may be certified by a government agency for use in prognosing the disease outcome of cancer patients and/or for designating a treatment modality.

Instructions for using the kit to perform one or more methods of the disclosure can be provided with the container, and can be provided in any fixed medium. The instructions may be located inside or outside the container or housing, and/or may be printed on the interior or exterior of any surface thereof.

### Amplification and Hybridization

Following sample collection and nucleic acid extraction, the nucleic acid portion of the sample comprising RNA that is or can be used to prepare the target polynucleotide(s) of interest can be subjected to one or more preparative reactions. These preparative reactions can include in vitro transcription (IVT), labeling, fragmentation, amplification and other reactions. mRNA can first be treated with reverse transcriptase and a primer to create cDNA prior to detection, quantitation and/or amplification; this can be done in vitro with purified mRNA or in situ, e.g., in cells or tissues affixed to a slide.

By “amplification” is meant any process of producing at least one copy of a nucleic acid, in this case an expressed RNA, and in many cases produces multiple copies. An amplification product can be RNA or DNA, and may include a complementary strand to the expressed target sequence. DNA amplification products can be produced initially through reverse translation and then optionally from further amplification reactions. The amplification product may include all or a portion of a target sequence, and may optionally be labeled. A variety of amplification methods are suitable for use, including polymerase-based methods and ligation-based methods. Exemplary amplification techniques include the polymerase chain reaction method (PCR), the lipase chain reaction (LCR), ribozyme-based methods, self-sustained sequence replication (3SR), nucleic acid sequence-based amplification (NASBA), the use of Q Beta replicase, reverse transcription, nick translation, and the like.

Asymmetric amplification reactions may be used to preferentially amplify one strand representing the target sequence that is used for detection as the target polynucleotide. In some cases, the presence and/or amount of the amplification product itself may be used to determine the expression level of a given target sequence. In other instances, the amplification product may be used to hybridize to an array or other substrate comprising sensor polynucleotides which are used to detect and/or quantitate target sequence expression.

The first cycle of amplification in polymerase-based methods typically forms a primer extension product complementary to the template strand. If the template is single-stranded RNA, a polymerase with reverse transcriptase activity is used in the first amplification to reverse transcribe the RNA to DNA, and additional amplification cycles can be performed to copy the primer extension products. The primers for a PCR must, of course, be designed to hybridize to regions in their corresponding template that can produce an amplifiable segment; thus, each primer must hybridize so that its 3′ nucleotide is paired to a nucleotide in its complementary template strand that is located 3′ from the 3′ nucleotide of the primer used to replicate that complementary template strand in the PCR.

The target polynucleotide can be amplified by contacting one or more strands of the target polynucleotide with a primer and a polymerase having suitable activity to extend the primer and copy the target polynucleotide to produce a full-length complementary polynucleotide or a smaller portion thereof. Any enzyme having a polymerase activity that can copy the target polynucleotide can be used, including DNA polymerases, RNA polymerases, reverse transcriptases, and enzymes having more than one type of polymerase or enzyme activity. The enzyme can be thermolabile or thermostable. Mixtures of enzymes can also be used. Exemplary enzymes include: DNA polymerases such as DNA Polymerase I (“Pol I”), the Klenow fragment of Pol I, T4, T7, Sequenase® T7, Sequenase® Version 2.0 T7, Tub, Taq. Tth, Pfic, Pfu, Tsp, Tfl, Tli and Pyrococcus sp GB-D DNA polymerases; RNA polymerases such as E coil, SP6, T3 and T7 RNA polymerases; and reverse transcriptases such as AMV, M-MuLV, MMLV, RNAse H MMLV (SuperScript®), SuperScript® II, ThermoScript®, HIV-1, and RAV2 reverse transcriptases. All of these enzymes are commercially available. Exemplary polymerases with multiple specificities include RAV2 and Tli (exo-) polymerases. Exemplary thermostable polymerases include Tub, Taq, Tth, Pfic, Pfu, Tvp, Ifl, Tli and Pyrococcus sp. GB-D DNA polymerases.

Suitable reaction conditions are chosen to permit amplification of the target polynucleotide, including pH, buffer, ionic strength, presence and concentration of one or more salts, presence and concentration of reactants and cofactors such as nucleotides and magnesium and/or other metal ions (e.g., manganese), optional cosolvents, temperature, thermal cycling profile for amplification schemes comprising a polymerase chain reaction, and may depend in part on the polymerase being used as well as the nature of the sample. Cosolvents include formamide (typically at from about 2 to about 10%), glycerol (typically at from about 5 to about 10%), and DMSO (typically at from about 0.9 to about 10%). Techniques may be used in the amplification scheme in order to minimize the production of false positives or artifacts produced during amplification. These include “touchdown” PCR, hot-start techniques, use of nested primers, or designing PCR primers so that they form stem-loop structures in the event of primer-dimer formation and thus are not amplified. Techniques to accelerate PCR can be used, for example centrifugal PCR, which allows for greater convection within the sample, and comprising infrared heating steps for rapid heating and cooling of the sample. One or more cycles of amplification can be performed. An excess of one primer can be used to produce an excess of one primer extension product during PCR; preferably, the primer extension product produced in excess is the amplification product to be detected. A plurality of different primers may be used to amplify different target polynucleotides or different regions of a particular target polynucleotide within the sample.

An amplification reaction can be performed under conditions which allow an optionally labeled sensor polynucleotide to hybridize to the amplification product during at least part of an amplification cycle. When the assay is performed in this manner, real-time detection of this hybridization event can take place by monitoring for light emission or fluorescence during amplification, as known in the art.

Where the amplification product is to be used for hybridization to an array or microarray, a number of suitable commercially available amplification products are available. These include amplification kits available from NuGEN, Inc. (San Carlos, CA), including the WT-Ovation™ System, WT-Ovation™ System v2, WT-Ovation™ Pico System, WT-Ovation™ FFPE Exon Module, WT-Ovation™ FFPE Exon Module RiboAmp and RiboAmp^{Plus} RNA Amplification Kits (MDS Analytical Technologies (formerly Arcturus) (Mountain View, CA), Genisphere, Inc. (Hatfield, PA), including the RampUp Plus™ and SenseAmp™ RNA Amplification kits, alone or in combination. Amplified nucleic acids may be subjected to one or more purification reactions after amplification and labeling, for example using magnetic beads (e.g., RNAClean magnetic beads, Agencourt Biosciences).

Multiple RNA biomarkers can be analyzed using real-time quantitative multiplex RT-PCR platforms and other multiplexing technologies such as GenomeLab GeXP Genetic Analysis System (Beckman Coulter, Foster City, CA), SmartCyclert 9600 or GeneXpert® Systems (Cepheid, Sunnyvale, CA), ABI 7900 HT Fast Real Time PCR system (Applied Biosystems, Foster City, CA), LightCycler® 480 System (Roche Molecular Systems, Pleasanton, CA), xMAP 100 System (Luminex, Austin, TX) Solexa Genome Analysis System (Illumina, Hayward, CA), OpenArray Real Time qPCR (BioTrove, Woburn, MA) and BeadXpress System (Illumina, Hayward, CA).

Detection and/or Quantification of Target Sequences

Any method of detecting and/or quantitating the expression of the encoded target sequences can in principle be used in the embodiments disclosed herein. The expressed target sequences can be directly detected and/or quantitated, or may be copied and/or amplified to allow detection of amplified copies of the expressed target sequences or its complement.

Methods for detecting and/or quantifying a target can include Northern blotting, sequencing, array or microarray hybridization, serial analysis of gene expression (SAGE), by enzymatic cleavage of specific structures (e.g., an Invader® assay, Third Wave Technologies, e.g. as described in U.S. Pat. Nos. 5,846,717, 6,090,543; 6,001,567; 5,985,557; and 5,994,069) and amplification methods, e.g. RT-PCR, including in a TaqMan® assay (PE Biosystems, Foster City, Calif., e.g. as described in U.S. Pat. Nos. 5,962,233 and 5,538,848), and may be quantitative or semi-quantitative, and may vary depending on the origin, amount and condition of the available biological sample. Combinations of these methods may also be used. For example, nucleic acids may be amplified, labeled and subjected to microarray analysis.

In some instances, target sequences may be detected by sequencing. Sequencing methods may comprise whole genome sequencing or exome sequencing. Sequencing methods such as Maxim-Gilbert, chain-termination, or high-throughput systems may also be used. Additional, suitable sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, and SOLiD sequencing.

Additional methods for detecting and/or quantifying a target include single-molecule sequencing (e.g., Helicos, PaeBio), sequencing by synthesis (e.g., Illumina, Ion Torrent), sequencing by ligation (e.g., ABI SOLID), sequencing by hybridization (e.g., Complete Genomics), in situ hybridization, bead-array technologies (e.g., Luminex xMAP, Illumina BeadChips), branched DNA technology (e.g., Panomics, Genisphere). Sequencing methods may use fluorescent (e.g., Illumina) or electronic (e.g., Ion Torrent, Oxford Nanopore) methods of detecting nucleotides.

### Reverse Transcription for ORT-PCR Analysis

Reverse transcription can be performed by any method known in the art. For example, reverse transcription may be performed using the Omniscript kit (Qiagen, Valencia, CA), Superscript III kit (Invitrogen, Carlsbad, CA), for RT-PCR. Target-specific priming can be performed in order to increase the sensitivity of detection of target sequences and generate target-specific cDNA.

### TaqMan® Gene Expression Analysis

TaqMan®RT-PCR can be performed using Applied Biosystems Prism (ABI) 7900 HT instruments in a 5 1.11 volume with target sequence-specific cDNA equivalent to 1 ng total RNA.

Primers and probes concentrations for TaqMan analysis are added to amplify fluorescent amplicons using PCR cycling conditions such as 95° C. for 10 minutes for one cycle, 95° C. for 20 seconds, and 60° C. for 45 seconds for 40 cycles. A reference sample can be assayed to ensure reagent and process stability. Negative controls (e.g., no template) should be assayed to monitor any exogenous nucleic acid contamination.

### Classification Arrays

The present disclosure contemplates that a probe set or probes derived therefrom may be provided in an array format. In the context of the present disclosure, an “array” is a spatially or logically organized collection of polynucleotide probes. An array comprising probes specific for a coding target, non-coding target, or a combination thereof may be used. Alternatively, an array comprising probes specific for two or more of transcripts of a target or a product derived thereof, can be used. Desirably, an array may be specific for 5, 10, 15, 20, 25, 30 or more of transcripts of a target of the disclosure. Expression of these targets may be detected alone or in combination with other transcripts. In some embodiments, an array is used which comprises a wide range of sensor probes for prostate-specific expression products, along with appropriate control sequences. In some instances, the array may comprise the Human Exon 1.0 ST Array (HuEx 1.0 ST, Affymetrix, Inc., Santa Clara, CA).

Typically the polynucleotide probes are attached to a solid substrate and are ordered so that the location (on the substrate) and the identity of each are known. The polynucleotide probes can be attached to one of a variety of solid substrates capable of withstanding the reagents and conditions necessary for use of the array. Examples include, but are not limited to, polymers, such as (poly)tetrafluoroethylene, (poly)vinylidenedifluoride, polystyrene, polycarbonate, polypropylene and polystyrene; ceramic; silicon; silicon dioxide; modified silicon; (fused) silica, quartz or glass; functionalized glass; paper, such as filter paper; diazotized cellulose; nitrocellulose filter; nylon membrane; and polyacrylamide gel pad. Substrates that are transparent to light are useful for arrays that may be used in an assay that involves optical detection.

Examples of array formats include membrane or filter arrays (for example, nitrocellulose, nylon arrays), plate arrays (for example, multiwell, such as a 24-, 96-, 256-, 384-, 864- or 1536-well, microtitre plate arrays), pin arrays, and bead arrays (for example, in a liquid “slurry”). Arrays on substrates such as glass or ceramic slides are often referred to as chip arrays or “chips.” Such arrays are well known in the art. In some embodiments of the present disclosure, the Cancer Prognostic array is a chip.

### Data Analysis

In some embodiments, one or more pattern recognition methods can be used in analyzing the expression level of target sequences. The pattern recognition method can comprise a linear combination of expression levels, or a nonlinear combination of expression levels. In some embodiments, expression measurements for RNA transcripts or combinations of RNA transcript levels are formulated into linear or non-linear models or algorithms (e.g., an ‘expression signature’) and converted into a likelihood score. This likelihood score may indicate the probability that a biological sample is from a patient who will benefit from immunotherapy. Additionally, a likelihood score may indicate the probability that a biological sample is from a patient who may exhibit no evidence of disease, who may exhibit systemic cancer, or who may exhibit biochemical recurrence. The likelihood score can be used to distinguish these disease states. The models and/or algorithms can be provided in machine readable format, and may be used to correlate expression levels or an expression profile with a disease state, and/or to designate a treatment modality for a patient or class of patients.

Assaying the expression level for a plurality of targets may comprise the use of an algorithm or classifier. Array data can be managed, classified, and analyzed using techniques known in the art. Assaying the expression level for a plurality of targets may comprise probe set modeling and data pre-processing. Probe set modeling and data pre-processing can be derived using the Robust Multi-Array (RMA) algorithm or variants GC-RMA, fRMA, Probe Logarithmic Intensity Error (PLIER) algorithm, or variant iterPLIER, or Single-Channel Array Normalization (SCAN) algorithm. Variance or intensity filters can be applied to pre-process data using the RMA algorithm, for example by removing target sequences with a standard deviation of <10 or a mean intensity of <100 intensity units of a normalized data range, respectively.

Alternatively, assaying the expression level for a plurality of targets may comprise the use of a machine learning algorithm. The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models.

The machine learning algorithms may also comprise an unsupervised learning algorithm. Examples of unsupervised learning algorithms may include artificial neural network, Data clustering, Expectation-maximization algorithm, Self-organizing map, Radial basis function network, Vector Quantization. Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering.

In some instances, the machine learning algorithms comprise a reinforcement learning algorithm. Examples of reinforcement learning algorithms include, but are not limited to, temporal difference learning, Q-learning and Learning Automata. Alternatively, the machine learning algorithm may comprise Data Pre-processing.

Preferably, the machine learning algorithms may include, but are not limited to, Average One-Dependence Estimators (AODE), Fisher's linear discriminant, Logistic regression, Perceptron, Multilayer Perceptron, Artificial Neural Networks, Support vector machines, Quadratic classifiers, Boosting, Decision trees, C4.5, Bayesian networks, Hidden Markov models, High-Dimensional Discriminant Analysis, and Gaussian Mixture Models. The machine learning algorithm may comprise support vector machines, Naïve Bayes classifier, k-nearest neighbor, high-dimensional discriminant analysis, or Gaussian mixture models. In some instances, the machine learning algorithm comprises Random Forests.

### Therapeutic Regimens

Diagnosing, predicting, or monitoring a status or outcome of a cancer may comprise treating a cancer or preventing a cancer progression. In addition, diagnosing, predicting, or monitoring a status or outcome of a cancer may comprise identifying or predicting which patients will be responders or non-responders to an anti-cancer therapy (e.g., immunotherapy). In some instances, diagnosing, predicting, or monitoring may comprise determining a therapeutic regimen. Determining a therapeutic regimen may comprise administering an immunotherapy. Alternatively, determining a therapeutic regimen may comprise modifying, recommending, continuing or discontinuing an anti-cancer regimen. In some instances, if the sample immune content levels are consistent with the immune content levels for a known disease or disease outcome, the immune content levels can be used to designate one or more treatment modalities (e.g., therapeutic regimens, such as immunotherapy or other anti-cancer regimen). An anti-cancer regimen may comprise one or more anti-cancer therapies. Examples of anti-cancer therapies include surgery, chemotherapy, radiation therapy, immunotherapy/biological therapy, and photodynamic therapy.

For example, a patient is selected for treatment with immunotherapy if the patient is identified as being likely to be responsive to immunotherapy based on an immune content score or levels, as described herein. Immunotherapy (sometimes called, biological therapy, biotherapy, biologic therapy, or biological response modifier (BRM) therapy) uses the body's immune system, either directly or indirectly, to fight cancer or to lessen the side effects that may be caused by some cancer treatments. Immunotherapies include interferons, interleukins, colony-stimulating factors, monoclonal antibodies, vaccines, immune cell-based therapy, gene therapy, and nonspecific immunomodulating agents. Sipuleucel-T is another immunotherapy that is useful in the methods of the disclosure. Sipuleucel-T, an autologous cellular immunological agent, is thought to work through APCs to stimulate T-cell immune response targeted against prostatic acid phosphatase (PAP), an antigen that is highly expressed in most prostate cancer cells.

Interferons (IFNs) are types of cytokines that occur naturally in the body. Interferon alpha, interferon beta, and interferon gamma are examples of interferons that may be used in cancer treatment.

Like interferons, interleukins (ILs) are cytokines that occur naturally in the body and can be made in the laboratory. Many interleukins have been identified for the treatment of cancer. For example, interleukin-2 (IL-2 or aldesleukin), interleukin 7, and interleukin 12 have may be used as an anti-cancer treatment. IL-2 may stimulate the growth and activity of many immune cells, such as lymphocytes, that can destroy cancer cells. Interleukins may be used to treat a number of cancers, including leukemia, lymphoma, and brain, colorectal, ovarian, breast, kidney and prostate cancers.

Colony-stimulating factors (CSFs) (sometimes called hematopoietic growth factors) may also be used for the treatment of cancer. Some examples of CSFs include, but are not limited to, G-CSF (filgrastim) and GM-CSF (sargramostim). CSFs may promote the division of bone marrow stem cells and their development into white blood cells, platelets, and red blood cells. Bone marrow is critical to the body's immune system because it is the source of all blood cells. Because anticancer drugs can damage the body's ability to make white blood cells, red blood cells, and platelets, stimulation of the immune system by CSFs may benefit patients undergoing other anti-cancer treatment, thus CSFs may be combined with other anti-cancer therapies, such as chemotherapy. CSFs may be used to treat a large variety of cancers, including lymphoma, leukemia, multiple myeloma, melanoma, and cancers of the brain, lung, esophagus, breast, uterus, ovary, prostate, kidney, colon, and rectum.

Another type of immunotherapy includes monoclonal antibodies (MOABs or MoABs). These antibodies may be produced by a single type of cell and may be specific for a particular antigen. To create MOABs, a human cancer cells may be injected into mice. In response, the mouse immune system can make antibodies against these cancer cells. The mouse plasma cells that produce antibodies may be isolated and fused with laboratory-grown cells to create “hybrid” cells called hybridomas. Hybridomas can indefinitely produce large quantities of these pure antibodies, or MOABs. MOABs may be used in cancer treatment in a number of ways. For instance, MOABs that react with specific types of cancer may enhance a patient's immune response to the cancer. MOABs can be programmed to act against cell growth factors, thus interfering with the growth of cancer cells.

MOABs may be linked to other anti-cancer therapies such as chemotherapeutics, radioisotopes (radioactive substances), other biological therapies, or other toxins. When the antibodies latch onto cancer cells, they deliver these anti-cancer therapies directly to the tumor, helping to destroy it. MOABs carrying radioisotopes may also prove useful in diagnosing certain cancers, such as colorectal, ovarian, and prostate.

Rituxan® (rituximab) and Herceptin® (trastuzumab) are examples of MOABs that may be used as a biological therapy. Rituxan may be used for the treatment of non-Hodgkin lymphoma. Herceptin can be used to treat metastatic breast cancer in patients with tumors that produce excess amounts of a protein called HER2. Alternatively, MOABs may be used to treat lymphoma, leukemia, melanoma, and cancers of the brain, breast, lung, kidney, colon, rectum, ovary, prostate, and other areas.

Cancer vaccines are another form of immunotherapy. Cancer vaccines may be designed to encourage the patient's immune system to recognize cancer cells. Cancer vaccines may be designed to treat existing cancers (therapeutic vaccines) or to prevent the development of cancer (prophylactic vaccines). Therapeutic vaccines may be injected in a person after cancer is diagnosed. These vaccines may stop the growth of existing tumors, prevent cancer from recurring, or eliminate cancer cells not killed by prior treatments. Cancer vaccines given when the tumor is small may be able to eradicate the cancer. On the other hand, prophylactic vaccines are given to healthy individuals before cancer develops. These vaccines are designed to stimulate the immune system to attack viruses that can cause cancer. By targeting these cancer-causing viruses, development of certain cancers may be prevented. For example, cervarix and gardasil are vaccines to treat human papilloma virus and may prevent cervical cancer. Therapeutic vaccines may be used to treat melanoma, lymphoma, leukemia, and cancers of the brain, breast, lung, kidney, ovary, prostate, pancreas, colon, and rectum. Cancer vaccines can be used in combination with other anti-cancer therapies.

Immune cell-based therapy is also another form of immunotherapy. Adoptive cell transfer may include the transfer of immune cells such as dendritic cells, T cells (e.g., cytotoxic T cells), or natural killer (NK) cells to activate a cytotoxic response or attack cancer cells in a patient. Autologous immune cell-based therapy involves the transfer of a patient's own immune cells after expansion in vitro.

Gene therapy is another example of a biological therapy. Gene therapy may involve introducing genetic material into a person's cells to fight disease. Gene therapy methods may improve a patient's immune response to cancer. For example, a gene may be inserted into an immune cell to enhance its ability to recognize and attack cancer cells. In another approach, cancer cells may be injected with genes that cause the cancer cells to produce cytokines and stimulate the immune system.

In some instances, biological therapy includes nonspecific immunomodulating agents. Nonspecific immunomodulating agents are substances that stimulate or indirectly augment the immune system. Often, these agents target key immune system cells and may cause secondary responses such as increased production of cytokines and immunoglobulins. Two nonspecific immunomodulating agents used in cancer treatment are bacillus Calmette-Guerin (BCG) and levamisole. BCG may be used in the treatment of superficial bladder cancer following surgery. BCG may work by stimulating an inflammatory, and possibly an immune, response. A solution of BCG may be instilled in the bladder. Levamisole is sometimes used along with fluorouracil (5-FU) chemotherapy in the treatment of stage III (Dukes' C) colon cancer following surgery. Levamisole may act to restore depressed immune function.

In addition, patients, especially those not identified as likely to benefit from immunotherapy, may be administered other cancer treatments such as, but not limited to, surgery, radiation therapy, chemotherapy, hormonal therapy, biologic therapy, or any combination thereof.

Surgical oncology uses surgical methods to diagnose, stage, and treat cancer, and to relieve certain cancer-related symptoms. Surgery may be used to remove the tumor (e.g., excisions, resections, debulking surgery), reconstruct a part of the body (e.g., restorative surgery), and/or to relieve symptoms such as pain (e.g., palliative surgery). Surgery may also include cryosurgery. Cryosurgery (also called cryotherapy) may use extreme cold produced by liquid nitrogen (or argon gas) to destroy abnormal tissue. Cryosurgery can be used to treat external tumors, such as those on the skin. For external tumors, liquid nitrogen can be applied directly to the cancer cells with a cotton swab or spraying device. Cryosurgery may also be used to treat tumors inside the body (internal tumors and tumors in the bone). For internal tumors, liquid nitrogen or argon gas may be circulated through a hollow instrument called a cryoprobe, which is placed in contact with the tumor. An ultrasound or MRI may be used to guide the cryoprobe and monitor the freezing of the cells, thus limiting damage to nearby healthy tissue. A ball of ice crystals may form around the probe, freezing nearby cells. Sometimes more than one probe is used to deliver the liquid nitrogen to various parts of the tumor. The probes may be put into the tumor during surgery or through the skin (percutaneously). After cryosurgery, the frozen tissue thaws and may be naturally absorbed by the body (for internal tumors), or may dissolve and form a scab (for external tumors).

Chemotherapeutic agents may also be used for the treatment of cancer. Examples of chemotherapeutic agents include alkylating agents, anti-metabolites, plant alkaloids and terpenoids, vinca alkaloids, podophyllotoxin, taxanes, topoisomerase inhibitors, and cytotoxic antibiotics. Cisplatin, carboplatin, and oxaliplatin are examples of alkylating agents. Other alkylating agents include mechlorethamine, cyclophosphamide, chlorambucil, ifosfamide. Alkylating agents may impair cell function by forming covalent bonds with the amino, carboxyl, sulfhydryl, and phosphate groups in biologically important molecules. Alternatively, alkylating agents may chemically modify a cell's DNA.

Anti-metabolites are another example of chemotherapeutic agents. Anti-metabolites may masquerade as purines or pyrimidines and may prevent purines and pyrimidines from becoming incorporated in to DNA during the “S” phase (of the cell cycle), thereby stopping normal development and division. Antimetabolites may also affect RNA synthesis. Examples of metabolites include azathioprine and mercaptopurine.

Alkaloids may be derived from plants and block cell division may also be used for the treatment of cancer. Alkyloids may prevent microtubule function. Examples of alkaloids are vinca alkaloids and taxanes. Vinca alkaloids may bind to specific sites on tubulin and inhibit the assembly of tubulin into microtubules (M phase of the cell cycle). The vinca alkaloids may be derived from the Madagascar periwinkle, Catharanthus roseus (formerly known as Vinca rosea). Examples of vinca alkaloids include, but are not limited to, vincristine, vinblastine, vinorelbine, or vindesine. Taxanes are diterpenes produced by the plants of the genus Taxus (yews). Taxanes may be derived from natural sources or synthesized artificially. Taxanes include paclitaxel (Taxol) and docetaxel (Taxotere). Taxanes may disrupt microtubule function. Microtubules are essential to cell division, and taxanes may stabilize GDP-bound tubulin in the microtubule, thereby inhibiting the process of cell division. Thus, in essence, taxanes may be mitotic inhibitors. Taxanes may also be radiosensitizing and often contain numerous chiral centers.

Alternative chemotherapeutic agents include podophyllotoxin. Podophyllotoxin is a plant-derived compound that may help with digestion and may be used to produce cytostatic drugs such as etoposide and teniposide. They may prevent the cell from entering the G1 phase (the start of DNA replication) and the replication of DNA (the S phase).

Topoisomerases are essential enzymes that maintain the topology of DNA. Inhibition of type I or type II topoisomerases may interfere with both transcription and replication of DNA by upsetting proper DNA supercoiling. Some chemotherapeutic agents may inhibit topoisomerases. For example, some type I topoisomerase inhibitors include camptothecins: irinotecan and topotecan. Examples of type II inhibitors include amsacrine, etoposide, etoposide phosphate, and teniposide.

Another example of chemotherapeutic agents is cytotoxic antibiotics. Cytotoxic antibiotics are a group of antibiotics that are used for the treatment of cancer because they may interfere with DNA replication and/or protein synthesis. Cytotoxic antibiotics include, but are not limited to, actinomycin, anthracyclines, doxorubicin, daunorubicin, valrubicin, idarubicin, epirubicin, bleomycin, plicamycin, and mitomycin.

In some instances, the anti-cancer treatment may comprise radiation therapy. Radiation can come from a machine outside the body (external-beam radiation therapy) or from radioactive material placed in the body near cancer cells (internal radiation therapy, more commonly called brachytherapy). Systemic radiation therapy uses a radioactive substance, given by mouth or into a vein that travels in the blood to tissues throughout the body.

External-beam radiation therapy may be delivered in the form of photon beams (either x-rays or gamma rays). A photon is the basic unit of light and other forms of electromagnetic radiation. An example of external-beam radiation therapy is called 3-dimensional conformal radiation therapy (3D-CRT). 3D-CRT may use computer software and advanced treatment machines to deliver radiation to very precisely shaped target areas. Many other methods of external-beam radiation therapy are currently being tested and used in cancer treatment. These methods include, but are not limited to, intensity-modulated radiation therapy (IMRT), image-guided radiation therapy (IGRT), Stereotactic radiosurgery (SRS), Stereotactic body radiation therapy (SBRT), and proton therapy.

Intensity-modulated radiation therapy (IMRT) is an example of external-beam radiation and may use hundreds of tiny radiation beam-shaping devices, called collimators, to deliver a single dose of radiation. The collimators can be stationary or can move during treatment, allowing the intensity of the radiation beams to change during treatment sessions. This kind of dose modulation allows different areas of a tumor or nearby tissues to receive different doses of radiation. IMRT is planned in reverse (called inverse treatment planning). In inverse treatment planning, the radiation doses to different areas of the tumor and surrounding tissue are planned in advance, and then a high-powered computer program calculates the required number of beams and angles of the radiation treatment. In contrast, during traditional (forward) treatment planning, the number and angles of the radiation beams are chosen in advance and computers calculate how much dose may be delivered from each of the planned beams. The goal of IMRT is to increase the radiation dose to the areas that need it and reduce radiation exposure to specific sensitive areas of surrounding normal tissue.

Another example of external-beam radiation is image-guided radiation therapy (IGRT). In IGRT, repeated imaging scans (CT, MRI, or PET) may be performed during treatment. These imaging scans may be processed by computers to identify changes in a tumor's size and location due to treatment and to allow the position of the patient or the planned radiation dose to be adjusted during treatment as needed. Repeated imaging can increase the accuracy of radiation treatment and may allow reductions in the planned volume of tissue to be treated, thereby decreasing the total radiation dose to normal tissue.

Tomotherapy is a type of image-guided IMRT. A tomotherapy machine is a hybrid between a CT imaging scanner and an external-beam radiation therapy machine. The part of the tomotherapy machine that delivers radiation for both imaging and treatment can rotate completely around the patient in the same manner as a normal CT scanner. Tomotherapy machines can capture CT images of the patient's tumor immediately before treatment sessions, to allow for very precise tumor targeting and sparing of normal tissue.

Stereotactic radiosurgery (SRS) can deliver one or more high doses of radiation to a small tumor. SRS uses extremely accurate image-guided tumor targeting and patient positioning. Therefore, a high dose of radiation can be given without excess damage to normal tissue. SRS can be used to treat small tumors with well-defined edges. It is most commonly used in the treatment of brain or spinal tumors and brain metastases from other cancer types. For the treatment of some brain metastases, patients may receive radiation therapy to the entire brain (called whole-brain radiation therapy) in addition to SRS. SRS requires the use of a head frame or other device to immobilize the patient during treatment to ensure that the high dose of radiation is delivered accurately.

Stereotactic body radiation therapy (SBRT) delivers radiation therapy in fewer sessions, using smaller radiation fields and higher doses than 3D-CRT in most cases. SBRT may treat tumors that lie outside the brain and spinal cord. Because these tumors are more likely to move with the normal motion of the body, and therefore cannot be targeted as accurately as tumors within the brain or spine, SBRT is usually given in more than one dose. SBRT can be used to treat small, isolated tumors, including cancers in the lung and liver. SBRT systems may be known by their brand names, such as the CyberKnife®.

In proton therapy, external-beam radiation therapy may be delivered by proton. Protons are a type of charged particle. Proton beams differ from photon beams mainly in the way they deposit energy in living tissue. Whereas photons deposit energy in small packets all along their path through tissue, protons deposit much of their energy at the end of their path (called the Bragg peak) and deposit less energy along the way. Use of protons may reduce the exposure of normal tissue to radiation, possibly allowing the delivery of higher doses of radiation to a tumor.

Other charged particle beams such as electron beams may be used to irradiate superficial tumors, such as skin cancer or tumors near the surface of the body, but they cannot travel very far through tissue.

Internal radiation therapy (brachytherapy) is radiation delivered from radiation sources (radioactive materials) placed inside or on the body. Several brachytherapy techniques are used in cancer treatment. Interstitial brachytherapy may use a radiation source placed within tumor tissue, such as within a prostate tumor. Intracavitary brachytherapy may use a source placed within a surgical cavity or a body cavity, such as the chest cavity, near a tumor. Episcleral brachytherapy, which may be used to treat melanoma inside the eye, may use a source that is attached to the eye. In brachytherapy, radioactive isotopes can be sealed in tiny pellets or “seeds.” These seeds may be placed in patients using delivery devices, such as needles, catheters, or some other type of carrier. As the isotopes decay naturally, they give off radiation that may damage nearby cancer cells. Brachytherapy may be able to deliver higher doses of radiation to some cancers than external-beam radiation therapy while causing less damage to normal tissue.

Brachytherapy can be given as a low-dose-rate or a high-dose-rate treatment. In low-dose-rate treatment, cancer cells receive continuous low-dose radiation from the source over a period of several days. In high-dose-rate treatment, a robotic machine attached to delivery tubes placed inside the body may guide one or more radioactive sources into or near a tumor, and then removes the sources at the end of each treatment session. High-dose-rate treatment can be given in one or more treatment sessions. An example of a high-dose-rate treatment is the MammoSite® system. Bracytherapy may be used to treat patients with breast cancer who have undergone breast-conserving surgery.

The placement of brachytherapy sources can be temporary or permanent. For permanent brachytherapy, the sources may be surgically sealed within the body and left there, even after all of the radiation has been given off. In some instances, the remaining material (in which the radioactive isotopes were sealed) does not cause any discomfort or harm to the patient. Permanent brachytherapy is a type of low-dose-rate brachytherapy. For temporary brachytherapy, tubes (catheters) or other carriers are used to deliver the radiation sources, and both the carriers and the radiation sources are removed after treatment. Temporary brachytherapy can be either low-dose-rate or high-dose-rate treatment. Brachytherapy may be used alone or in addition to external-beam radiation therapy to provide a “boost” of radiation to a tumor while sparing surrounding normal tissue.

In systemic radiation therapy, a patient may swallow or receive an injection of a radioactive substance, such as radioactive iodine or a radioactive substance bound to a monoclonal antibody. Radioactive iodine (131I) is a type of systemic radiation therapy commonly used to help treat cancer, such as thyroid cancer. Thyroid cells naturally take up radioactive iodine. For systemic radiation therapy for some other types of cancer, a monoclonal antibody may help target the radioactive substance to the right place. The antibody joined to the radioactive substance travels through the blood, locating and killing tumor cells. For example, the drug ibritumomab tiuxetan (Zevalin®) may be used for the treatment of certain types of B-cell non-Hodgkin lymphoma (NHL). The antibody part of this drug recognizes and binds to a protein found on the surface of B lymphocytes. The combination drug regimen of tositumomab and iodine I 131 tositumomab (Bexxart) may be used for the treatment of certain types of cancer, such as NHL. In this regimen, nonradioactive tositumomab antibodies may be given to patients first, followed by treatment with tositumomab antibodies that have 131I attached. Tositumomab may recognize and bind to the same protein on B lymphocytes as ibritumomab. The nonradioactive form of the antibody may help protect normal B lymphocytes from being damaged by radiation from 131I.

Some systemic radiation therapy drugs relieve pain from cancer that has spread to the bone (bone metastases). This is a type of palliative radiation therapy. The radioactive drugs samarium-153-lexidronam (Quadramet®) and strontium-89 chloride (Metastron®) are examples of radiopharmaceuticals may be used to treat pain from bone metastases.

Photodynamic therapy (PDT) is an anti-cancer treatment that may use a drug, called a photosensitizer or photosensitizing agent, and a particular type of light. When photosensitizers are exposed to a specific wavelength of light, they may produce a form of oxygen that kills nearby cells. A photosensitizer may be activated by light of a specific wavelength. This wavelength determines how far the light can travel into the body. Thus, photosensitizers and wavelengths of light may be used to treat different areas of the body with PDT.

In the first step of PDT for cancer treatment, a photosensitizing agent may be injected into the bloodstream. The agent may be absorbed by cells all over the body but may stay in cancer cells longer than it does in normal cells. Approximately 24 to 72 hours after injection, when most of the agent has left normal cells but remains in cancer cells, the tumor can be exposed to light. The photosensitizer in the tumor can absorb the light and produces an active form of oxygen that destroys nearby cancer cells. In addition to directly killing cancer cells, PDT may shrink or destroy tumors in two other ways. The photosensitizer can damage blood vessels in the tumor, thereby preventing the cancer from receiving necessary nutrients. PDT may also activate the immune system to attack the tumor cells.

The light used for PDT can come from a laser or other sources. Laser light can be directed through fiber optic cables (thin fibers that transmit light) to deliver light to areas inside the body. For example, a fiber optic cable can be inserted through an endoscope (a thin, lighted tube used to look at tissues inside the body) into the lungs or esophagus to treat cancer in these organs. Other light sources include light-emitting diodes (LEDs), which may be used for surface tumors, such as skin cancer. PDT is usually performed as an outpatient procedure. PDT may also be repeated and may be used with other therapies, such as surgery, radiation, or chemotherapy.

Extracorporeal photopheresis (ECP) is a type of PDT in which a machine may be used to collect the patient's blood cells. The patient's blood cells may be treated outside the body with a photosensitizing agent, exposed to light, and then returned to the patient. ECP may be used to help lessen the severity of skin symptoms of cutaneous T-cell lymphoma that has not responded to other therapies. ECP may be used to treat other blood cancers, and may also help reduce rejection after transplants.

Additionally, photosensitizing agent, such as porfimer sodium or Photofrin® may be used in PDT to treat or relieve the symptoms of esophageal cancer and non-small cell lung cancer. Porfimer sodium may relieve symptoms of esophageal cancer when the cancer obstructs the esophagus or when the cancer cannot be satisfactorily treated with laser therapy alone. Porfimer sodium may be used to treat non-small cell lung cancer in patients for whom the usual treatments are not appropriate, and to relieve symptoms in patients with non-small cell lung cancer that obstructs the airways. Porfimer sodium may also be used for the treatment of precancerous lesions in patients with Barrett esophagus, a condition that can lead to esophageal cancer.

Laser therapy may use high-intensity light to treat cancer and other illnesses. Lasers can be used to shrink or destroy tumors or precancerous growths. Lasers are most commonly used to treat superficial cancers (cancers on the surface of the body or the lining of internal organs) such as basal cell skin cancer and the very early stages of some cancers, such as cervical, penile, vaginal, vulvar, and non-small cell lung cancer.

Lasers may also be used to relieve certain symptoms of cancer, such as bleeding or obstruction. For example, lasers can be used to shrink or destroy a tumor that is blocking a patient's trachea (windpipe) or esophagus. Lasers also can be used to remove colon polyps or tumors that are blocking the colon or stomach.

Laser therapy is often given through a flexible endoscope (a thin, lighted tube used to look at tissues inside the body). The endoscope is fitted with optical fibers (thin fibers that transmit light). It is inserted through an opening in the body, such as the mouth, nose, anus, or vagina. Laser light is then precisely aimed to cut or destroy a tumor.

Laser-induced interstitial thermotherapy (LITT), or interstitial laser photocoagulation, also uses lasers to treat some cancers. LITT is similar to a cancer treatment called hyperthermia, which uses heat to shrink tumors by damaging or killing cancer cells. During LITT, an optical fiber is inserted into a tumor. Laser light at the tip of the fiber raises the temperature of the tumor cells and damages or destroys them. LITT is sometimes used to shrink tumors in the liver.

Laser therapy can be used alone, but most often it is combined with other treatments, such as surgery, chemotherapy, or radiation therapy. In addition, lasers can seal nerve endings to reduce pain after surgery and seal lymph vessels to reduce swelling and limit the spread of tumor cells.

Lasers used to treat cancer may include carbon dioxide (CO₂) lasers, argon lasers, and neodymium:yttrium-aluminum-garnet (Nd:YAG) lasers. Each of these can shrink or destroy tumors and can be used with endoscopes. CO₂ and argon lasers can cut the skin's surface without going into deeper layers. Thus, they can be used to remove superficial cancers, such as skin cancer. In contrast, the Nd:YAG laser is more commonly applied through an endoscope to treat internal organs, such as the uterus, esophagus, and colon. Nd:YAG laser light can also travel through optical fibers into specific areas of the body during LITT. Argon lasers are often used to activate the drugs used in PDT.

Immune content levels can be grouped so that information obtained about the set of targets in the group can be used to make or assist in making a clinically relevant judgment such as a diagnosis, prognosis, or treatment choice. For example, immune content targets can consist of one or more targets from the following list of immune content targets: plasma cells, tertiary lymphoid structure activity, IgG expression, interferon gamma (IFNG) signaling, and/or natural killer (NK) cell activity.

A patient report is also provided comprising a representation of measured levels of a plurality of immune content targets in a biological sample from the patient, wherein the representation comprises levels of immune content targets corresponding to any one, two, three, four, or five of the targets described herein, or a combination thereof. In some embodiments, the representation of the measured expression level(s) may take the form of a linear or nonlinear combination of levels of the target of interest. The patient report may be provided in a machine (e.g., a computer) readable format and/or in a hard (paper) copy. The report can also include standard measurements of levels of said plurality of targets from one or more sets of patients with known disease status and/or outcome. The report can be used to inform the patient and/or treating physician of the levels of the targets, the likely medical diagnosis and/or implications, and may recommend immunotherapy for the patient.

Also provided are representations of the immune content scores useful for treating, diagnosing, prognosticating, and otherwise assessing prostate cancer. In some embodiments, these profile representations are reduced to a medium that can be automatically read by a machine such as computer readable media (magnetic, optical, and the like). The articles can also include instructions for assessing the immune content scores in such media. For example, the articles may comprise a readable storage form having computer instructions for comparing immune content scores of the targets described herein. The articles may also have immune content scores digitally recorded therein so that they may be compared with target data from patient samples. Alternatively, the profiles can be recorded in different representational format. A graphical recordation is one such format. Clustering algorithms can assist in the visualization of such data.

### Clinical Associations and Patient Outcomes

Molecular subtypes of the present disclosure have distinct clinical associations. Clinical associations that correlate to molecular subtypes include, for example, preoperative serum PSA, Gleason score (GS), extraprostatic extension (EPE), surgical margin status (SM), lymph node involvement (LNI), and seminal vesicle invasion (SVI).

For example, increasing plasma cell content can be correlated with prolonged metastasis-free and disease-free survival. In particular, high IgG expression is associated with longer metastasis-free survival (see Example 1). Increased NK activity is associated with longer duration to metastatic disease following radical prostatectomy in patients with prostate cancer.

In some embodiments, immune content scores of the present disclosure are used to predict patient outcomes such as biochemical recurrence (BCR), metastasis (MET) and prostate cancer death (PCSM) after radical prostatectomy.

### Prediction of Treatment Response to Immunotherapy

Immune content scores can be utilized to predict whether or not a cancer patient will benefit from immunotherapy. Immune content scores can be determined by detecting levels of plasma cells, tertiary lymphoid structure activity, IgG expression, interferon gamma (IFNG) signaling, and/or natural killer (NK) cell activity (see Methods). Accordingly, immune content scores can be used to detect increased levels of plasma cells, tertiary lymphoid structure activity. IgG expression, interferon gamma (IFNG) signaling, and/or natural killer (NK) cell activity in a sample from a subject, which indicates that the subject is likely to benefit from immunotherapy. The use of immune content scores allows the identification of subjects with high levels of immune content in prostate cancer, who are likely to benefit from immunotherapy as well as subjects with low immune content in prostate cancer, who may benefit from another anti-cancer therapy.

Patients with little or no detectable immune content tend to fail treatment with immunotherapy and are better candidates for other anti-cancer treatment options.

Below are examples of embodiments for carrying out the present disclosure. The examples are offered for illustrative purposes only, and are not intended to limit the scope of the present disclosure in any way.

Efforts have been made to ensure accuracy with respect to numbers used (e.g., amounts, temperatures, etc.), but some experimental error and deviation should, of course, be allowed for.

### EXAMPLES

### Example 1: Development and Validation of Immune Content Scores to Identify Prostate Tumors that are Likely to Respond to Immunotherapy

Immune content scores to identify tumors that will likely be responsive to immunotherapy were developed as follows. Three cohorts of prostate cancer (PC) tumors were derived to first discover varying immunogenic signatures by racial ancestry. The discovery cohort was comprised of two groups of patients who underwent radical prostatectomy and no additional treatment until metastatic recurrence at Johns Hopkins Medical Institute (JHMI). The first group consisted of 355 intermediate- or high-risk patients treated between 1995 and 2005, of which 33 were Black. The second group consisted 143 Black men treated from 2006 to 20107 for a total of 355 White men and 176 Black men. To prepare for matching, 6 (1%) men were excluded due to missing data on pathological stage. All men with grade group 1 disease were excluded given the disproportionate numbers among Black men (24 vs 6; 30 total, 6%). Finally, the cohort was randomly sorted before using the “matchit” function from the “Matchit” R package to derive a final grade- and stage-matched cohort of 300 men, 150 each of Black and White men. Race was defined by self-identification in this cohort.

The first validation cohort was generated from publicly available data within The Cancer Genome Atlas (TCGA) PRAD dataset (n=468). Race in this cohort was defined by genetic ancestry (410 White and 58 Black). Expression, DNA methylation, and clinical data from TCGA were downloaded via eBioPortal. Pre-operative prostate-specific antigen levels for TCGA were downloaded from the Broad Institute TCGA Genome Data Analysis Center. Because there were so few Black men represented in TCGA general trends in TCGA as validation for findings from JHMI. To further confirm findings from JHMI, we used a second, large retrospective cohort with whole transcriptomic data (DVA; n=538). Race was patient-identified in this cohort (236 White and 302 Black). This group was comprised of genome-wide expression profiles from formalin-fixed paraffin-embedded prostate tumors from radical prostatectomy between 1989 and 2016. Investigation in our discovery cohort were exploratory and all analyses in the validation cohorts were confirmatory. Characteristics of our discovery and validation cohorts can be seen in Tables 1-3 below. Following the discovery and validation of the findings regarding increased quantities of B-lineage and plasma cells in PC-B, a fourth cohort for DNA-methylation-based and histologic validation was leveraged. This cohort consisted of 135 PC-B grade-matched to 135 PC-W who underwent radical prostatectomy. Complete description of this cohort can be found in Kaur et al. Measurements from tumors were from distinct samples without any repeated measurements. Finally, data from biopsies of 118 mCRPC tumors were downloaded from eBioPortal. Self-identified race was provided for 109 patients (Table 4).

TABLE 1
Johns Hopkins Medical Institute cohort

| | White | Black | |
|---|---|---|---|
| Characteristic | n = 150 | n = 150 | p |
| Age | | | 0.015 |
| Mean (SD) | 60.0 (5.7) | 58.3 (6.2) | |
| Grade Group | | | 0.060 |
| 2 | 49 (32.7%) | 42 (28%) | |
| 3 | 42 (28%) | 60 (40%) | |
| 4 | 24 (16%) | 27 (18%) | |
| 5 | 35 (23.3%) | 21 (14%) | |
| PSA, ng/mL | | | 0.131 |
| Mean (SD) | 9.8 (6.7) | 10.8 (8.1) | 1.0 |
| Unknown | 0 (0%) | 2 (1.3%) | |
| Organ Confined | | | 0.9 |
| Yes | 110 (73.3%) | 110 (73.3%) | |
| No | 40 (26.7%) | 40 (26.7%) | |
| Margin + | | | 0.145 |
| No | 110 (73.3%) | 96 (64%) | |
| Yes | 40 (26.7%) | 52 (34.7%) | |
| Unknown | 0 (0%) | 2 (1.3%) | |

Abbreviations: SD, standard deviation; PSA, prostate specific antigen

TABLE 2
The Cancer Genome Atlas cohort

| | White | Black | |
|---|---|---|---|
| Characteristic | n = 410 | n = 58 | p |
| Age | | | <0.001 |
| Mean (SD) | 61.7 (6.5) | 57.3 (7.2) | |
| Grade Group | | | <0.001 |
| 1 | 30 (7.3%) | 12 (20.7%) | |
| 2 | 116 (28.3%) | 26 (44.8%) | |
| 3 | 84 (20.5%) | 12 (20.7%) | |
| 4 | 57 (13.9%) | 2 (3.4%) | |
| 5 | 123 (30%) | 6 (10.3%) | |
| PSA, ng/mL | | | 0.779 |
| Mean (SD) | 10.9 (12.4) | 11.4 (11.5) | |
| Organ Confined | | | 0.024 |
| Yes | 149 (36.3%) | 31 (53.4%) | |
| No | 254 (62%) | 27 (46.6%) | |
| Unknown | 7 (1.7%) | 0 (0%) | |
| Margin + | | | 0.7 |
| No | 273 (66.6%) | 38 (65.5%) | |
| Yes | 127 (31%) | 15 (25.9%) | |
| Unknown | 10 (2.4%) | 5 (8.6%) | |

Abbreviations: SD, standard deviation; PSA, prostate specific antigen

TABLE 3
Durham Veterans Affairs cohort

| | White | Black | |
|---|---|---|---|
| Characteristic | n = 236 | n = 302 | p |
| Age | | | <0.001 |
| Mean (SD) | 62.7 (5.7) | 59.9 (6.0) | |
| Grade Group | | | 0.3 |
| 1 | 35 (14.8%) | 30 (9.9%) | |
| 2 | 137 (58.1%) | 191 (63.2%) | |
| 3 | 33 (14%) | 48 (15.9%) | |
| 4 | 11 (4.7%) | 16 (5.3%) | |
| 5 | 20 (8.5%) | 17 (5.6%) | |
| PSA, ng/mL | | | 0.021 |
| Mean (SD) | 8.8 (6.7) | 10.6 (10.3) | |
| Organ Confined | | | 0.023 |
| Yes | 119 (50.4%) | 183 (60.6%) | |
| No | 117 (49.6%) | 119 (39.4%) | |
| Margin + | | | 0.145 |
| No | 31 (13.1%) | 45 (14.9%) | |
| Yes | 205 (86.9%) | 257 (85.1%) | |
| Adjuvant hormonal therapy | | | 0.6 |
| No | 177 (75%) | 219 (72.5%) | |
| Yes | 59 (25%) | 82 (27.2%) | |
| Unknown | 0 (0%) | 1 (0.3%) | |
| Adjuvant radiotherapy | | | 0.142 |
| No | 132 (55.9%) | 148 (49%) | |
| Yes | 104 (44.1%) | 153 (50.7%) | |
| Unknown | 0 (0%) | 1 (0.3%) | |

Abbreviations: SD, standard deviation; PSA, prostate specific antigen

TABLE 4
Metastatic castrate resistant prostate cancer cohort

| Characteristic | n (%) | |
|---|---|---|
| All | 118 | (100) |

Age

| | | |
|---|---|---|
| Mean (SD) | 67.2 | (8.3) |

Race

| | | |
|---|---|---|
| White | 102 | (86.4) |
| Black | 7 | (5.9) |
| Unknown/Other | 9 | (7.6) |

Biopsy site

| | | |
|---|---|---|
| Visceral | 22 | (18.6) |
| Lymph node | 50 | (42.4) |
| Skeletal | 31 | (26.3) |
| Unknown/Other | 15 | (12.7) |

Prior taxane treatment

| | | |
|---|---|---|
| Yes | 44 | (37.3) |
| No | 71 | (60.2) |
| Unknown | 3 | (2.5) |

Prior abiraterone or
enzalutamide treatment

| | | |
|---|---|---|
| Yes | 54 | (45.8) |
| No | 61 | (51.7) |
| Unknown | 3 | (2.5) |

Abbreviation: SD, standard deviation

### Gene Expression Profiling and Genomic Data

Expression profiling for tumors from JHMI and the DVA were conducted in a Clinical Laboratory Improvement Amendments (CLIA)-certified laboratory facility (Decipher Biosciences, San Diego, CA, USA). All tumors underwent central pathology review and at least 0.5 mm² of tumor with 60/o tumor cellularity were required for sampling. RNeasy FFPE (Qiagen, Valencia, CA) was used for RNA extraction and purification. Ovation WTA FFPE system (NuGen, San Carlos, CA) was used for amplification and labeling of RNA, which was then hybridized to Human Exon 1.0 ST GeneChips (Thermo-Fisher, Carlsbad, CA). Affymetrix Power Tools were used for quality control. Finally, the Single Channel Array Normalization (SCAN) algorithm was used for normalization. TCGA and mCRPC expression data were quantile matched to the microarray platform such that all signatures developed on the microarray platform could be applied. All genomic and expression data for TCGA and mCRPC was acquired from eBioPortal.

### Tumor Immune Cell Content

Tumor immune and stromal content were calculated based on RNA expression using methods from Yoshihara. K. et al. Nature Communications 4, 1-11 (2013). The final immune content score was the derived immune score plus the constant of the minimum score in each cohort plus one. This was done so that when we would apply the proportions of the deconvolution of tumor-invading lymphocytes the final quantities of tumor-invading lymphocytes would be positive. Tumor-invading lymphocytes were deconvoluted using the MySort tool implemented in a R function (see Chen, S.-H. et al. BMC Bioinformatics 19, 154 (2018)). This produced a proportion of 2I different immune cells within each tumor, which was multiplied by the estimated immune content to derive a quantity of each cell type that could be compared between samples within the same cohort. A second RNA expression-based method for immune cell deconvolution, Microenvironment Cell Populations-counter (MCP-counter), was applied to confirm findings from MySort.

As an orthogonal approach to deconvolute the immune TME, the DNA methylation-based tool methyCIBERSORT was applied to TCGA and Kaur et al (Kaur, H. B. et al. Mod Pathol 31, 1539-1552 (2018) and Chakravarthy, A. et al. Nature Communications 9, 1-13 (2018)). The whole methylomes of these cohorts were investigated through the Infinium MethylationEPIC array platform. The minfi package in R was used to determine the quality of methylation experiments and to derive single probe scores per tumor sample. For methylCIBERSORT analysis, raw probe scores were normalized using the preprocessNoob function in minfi.

### ERG+/− and PTEN Loss Signatures

A validated transcriptome-based signature with 95% accuracy for predicting ERG+/− status as described by Tomlins et al. was applied to JHMI and DVA to assess the interaction between ERG+/− and race and race-based differences in the TME (Tomlins, S. A. et al. European Urology 68, 555-567 (2015). To the DVA tumors, an expression-based signature predictive of PTEN loss was applied as well.

### Tertiary Lymphoid Structure Signature

Using a list of tertiary lymphoid structure-hallmark genes, (Cabrita, R. et al. Nature 577, 561-565 (2020) and Dieu-Nosjean, M.-C., et al. Trends in Immunology 35, 571-580 (2014)), a signature to reflect the activity and presence of tertiary lymphoid structures within the TME was generated as the geometric mean expression of seven genes (CCR7, CXCR5, SELL, LAMP3, CCL19, CCL21, and CXCL13).

### Immunohistochemistry and Image Analysis

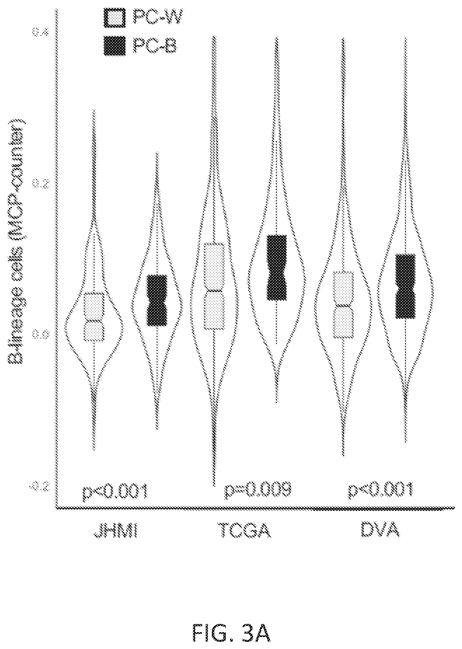

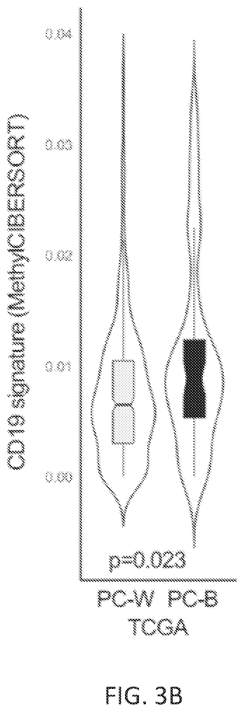

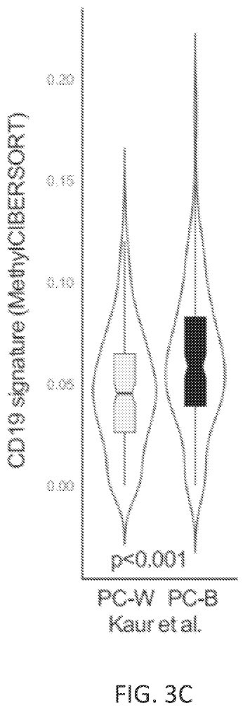

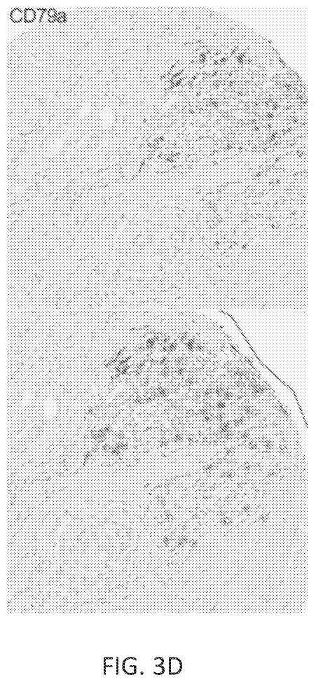

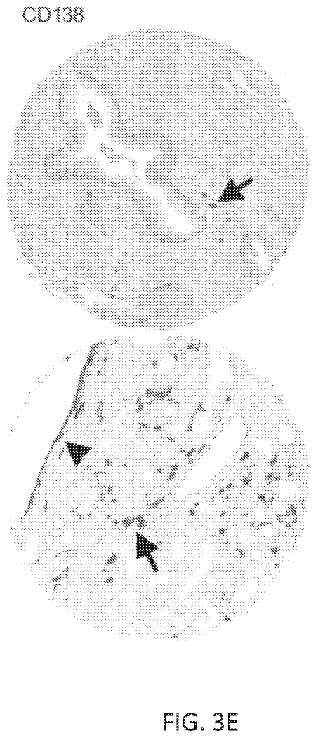

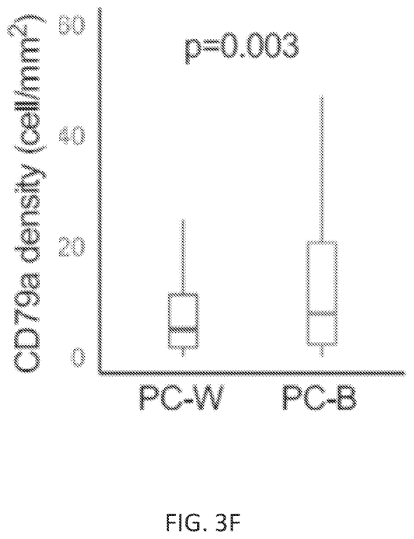

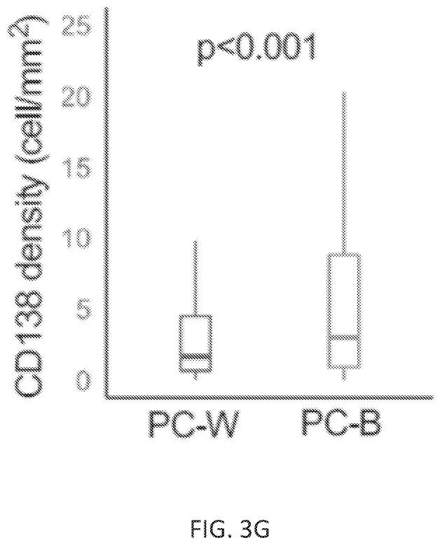

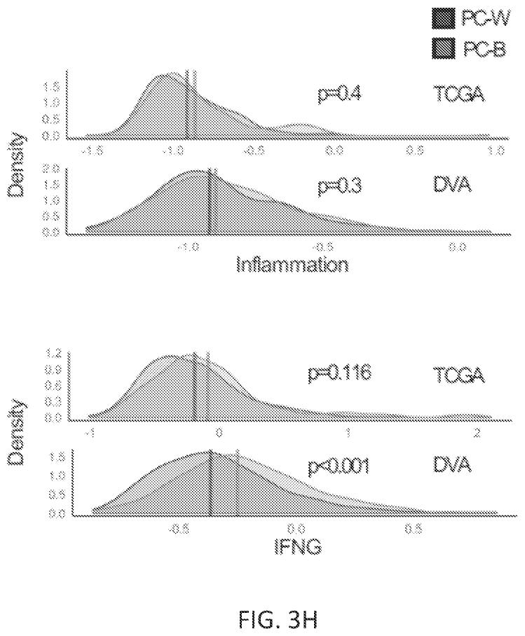

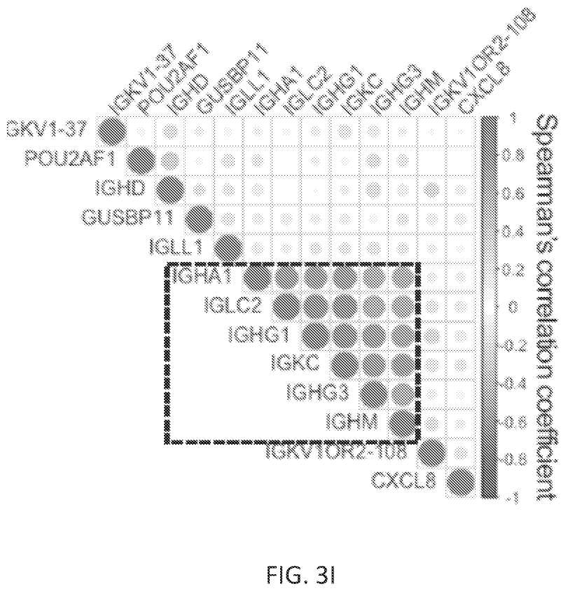

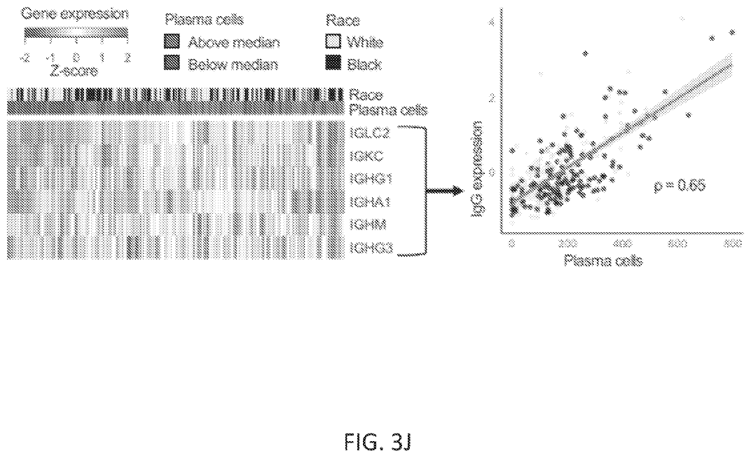

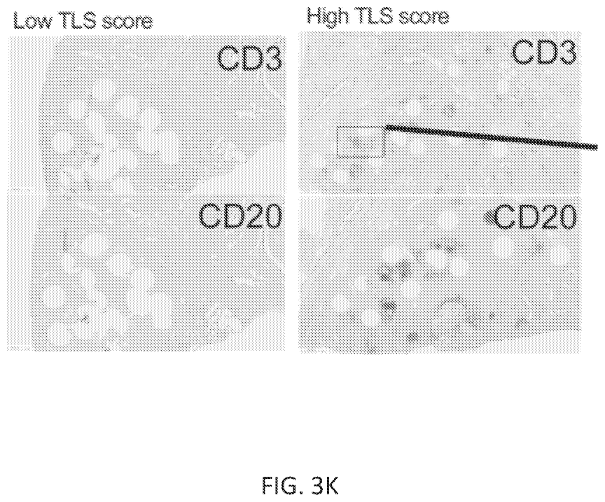

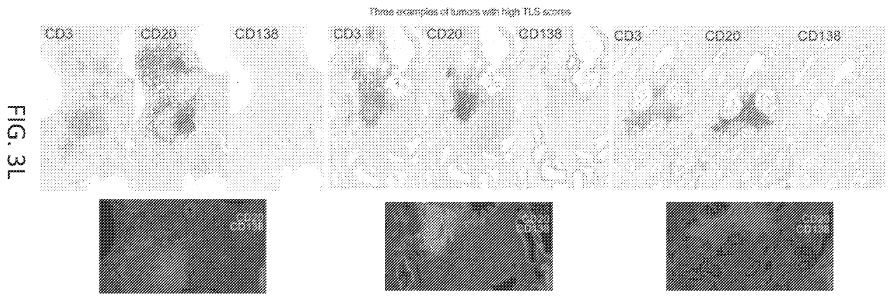

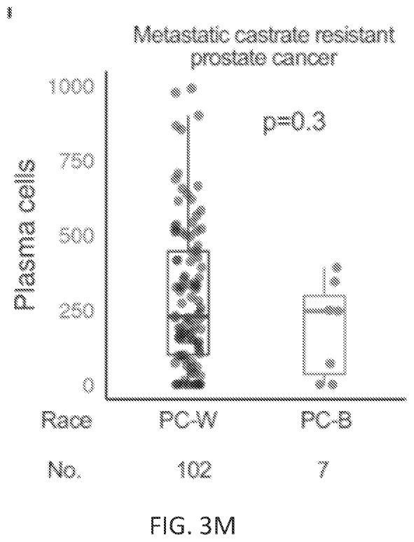

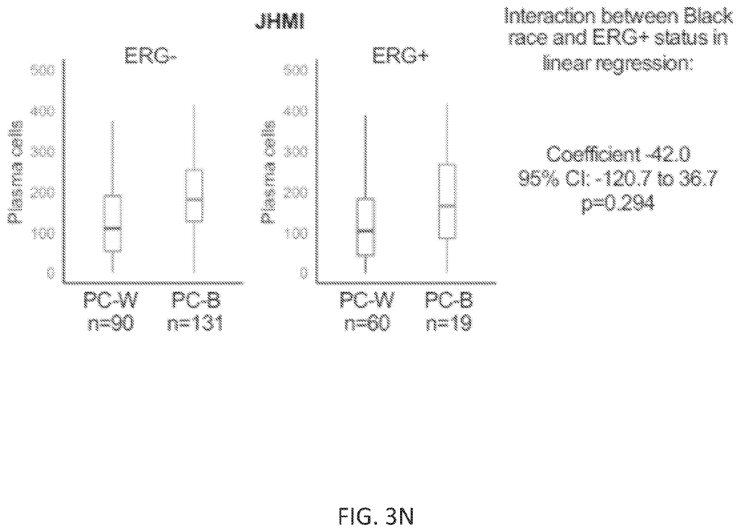

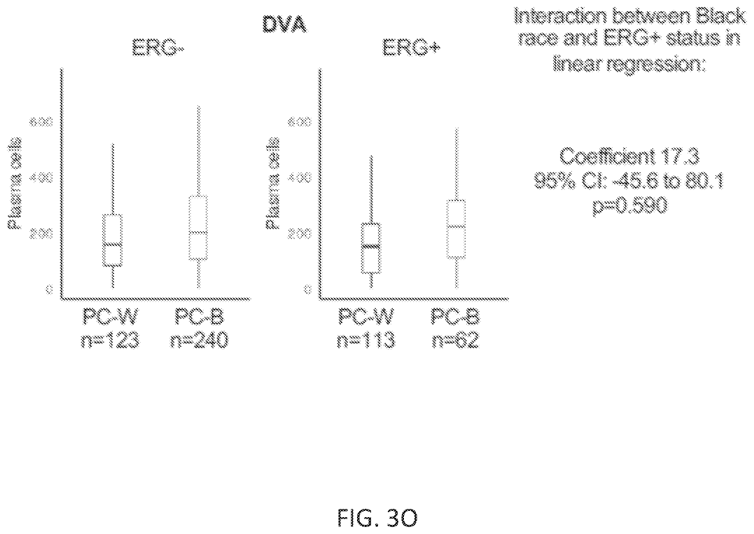

Immunohistochemistry for CD79a, a pan-B-cell membrane protein, (Kroeger, D. R., et al. Clin Cancer Res 22, 3005-3015 (2016)) and CD138/syndecan-1, a marker of plasma cells, within the Kaur et al. cohort was conducted using mouse monoclonal antibodies (JCB117, CellMarque, Rocklin, CA and B-A38, Ventana/Roche, respectively) on the Ventana Benchmark platform in a CLIA-accredited laboratory. For CD79a, cell densities were determined using QuPath digital image analysis algorithms to count the number of CD79a+ cells within each 0.6 mm diameter tissue microarray punch of tumor (average of four spots sampled per case), normalized by the total mm² of tissue per, and taking the mean across the four tumor cores for each patient. In the case of CD138, patchy expression of CD138 in the benign prostate epithelium (predominantly basal cells) was observed as well as in a small subset of tumor cells, making automated digital image quantification of CD138+ cells impossible (see arrowhead in FIG. 3E). To circumvent this issue, a blinded manual quantification of CD138+ cell density was performed, counting all individual cells positive for CD138 in each 0.6 mm diameter tissue core of tumors (average of four spots sampled per case), normalized by the total mm² of tissue analyzed for each core calculated in QuPath as above and taking the mean across the four tumor cores for each patient.

As a qualitative means of assessing the expression-based signature for TLS, five tumors with high TLS signature scores (highest pentile) were randomly chosen and immunostained for CD3 (Rabbit polyclonal, A0452, Dako), CD20 (mouse monoclonal, L26, Ventana/Roch), and CD138 (Mouse monoclonal, B-A38, Ventana/Roche) on adjacent slides using the Ventana Benchmark system in a CLIA-accredited lab. These images were merged and pseudocolored to demonstrate the spatial relational between cell types. In each, discrete lymphoid aggregates of T- and B-cells, with scattered adjacent CD138+ cells, are demonstrated suggesting the presence of TLS (FIG. 3L). Three tumors in the lowest pentile of TLS signature scores were also randomly chosen and immunostained for CD20 and CD3 to qualitatively demonstrate a lack of lymphoid aggregates relative to high TLS tumors. PTEN status in JHMI samples were based on immunohistochemistry as previously described (Haney Nora M. et al. Journal of Urology 203, 344-350(2020)).

### Markers of Tumor Immune Susceptibility and Activity

A tumor lymphocyte evasion score was derived and defined by the expression of genes from cell types that are capable of evading lymphocyte infiltration (cancer-associated fibroblasts [CAFs], myeloid-derived suppressor cells [MDSCs], and M2 subtype of tumor-associated macrophages [TAMs]) as described by Jiang et al. (“exclusion” score in previous publication) (Jiang, P. et al. Nat Med 24, 1550-1558 (2018)). From the Hallmark Gene Set Collection, a measure of genes upregulated during chronic and acute inflammation (HALLMARK_INFLAMMATORY_RESPONSE) and interferongamma (IFNG; HALLMARK_INTERFERON_GAMMA_RESPONSE) was derived (Liberzon, A. et al. Cell Syst. 1, 417-425 (2015)). Increased expression of this IFNG signature was previously shown to be enriched in tumors that responded to anti-CTLA-4 systemic therapy in men with advanced PC (Subudhi, S. K. et al. Science Translational Medicine 12, (2020)).

Within TCGA, neoantigen burden data were downloaded from The Cancer Immune Atlas project (https://tcia.at/) which was calculated using the OptiType pipeline (Szolek, A. et al. Bioinformatics 30, 3310-3316 (2014)). Data were available for 420 of the 468 in the final cohort. Fraction genome altered was defined as the length of segments with log 2 or linear copy number alteration value larger than 0.2 divided by the length of all segments measured as available in eBioPortal (Cerami, E. et al. Cancer Discov 2, 401-404(2012); Gao, J. et al. Sci. Signal. 6, pl1-pl1 (2013)).

### IG Expression Metagene

An unsupervised hierarchal clustering was applied to a gene-set of proteins related to IgG components and expression originally assessed in breast cancer (Rody, A. et al. Breast Cancer Research 11, R15 (2009)). Six IgG genes were highly correlated with each other in PC tumors from JHMI (IGLC2, IGKC, IGHG1, IGHA1, IGHM, and IGHG3; FIG. 3I), each of which were independent of the signature matrix used for immune cell deconvolution signature matrix used with MySort. The geometric mean expression of these genes comprised our IgG expression metagene, a measure of plasma cell activity.

### T-Cell and NK Activity Signatures

Cytotoxic T-cell (CD8+) activity was measured as the geometric mean expression of GZMA and PRF1 as described by Rooney et al. (Cell 160, 48-61 (2015)). Because NK cells have overlapping gene expression with CD8+ T-cells, we sought to derive a metagene more specific to NK cells activation. As described by Cursons et al. (Cancer Immunol Res 7, 1162-1174 (2019)), we represented NK activity as the geometric mean expression of eight genes expressed during NK activation (ILI5, XCL1, XCL2, CCL5, FLT3LG, GZMA, GZMB, and FASLG).

### Clinical Outcome Measurements

Patients from JHMI were followed until the development of metastatic disease following radical prostatectomy. Notably, in this cohort, no patients received any adjuvant treatment following surgery. Metastatic disease development was assessed with imaging using computed tomography or bone scan following biochemical recurrence, as defined by a rise in PSA>0.2 ng/mL. In TCGA, patients were followed until any disease recurrence which included the earlier of development of metastatic disease as well as any biochemical recurrence. Included in multivariable analyses for metastasis-free survival was a validated genomic risk score for developing metastatic disease following treatment for localized prostatectomy based on transcriptomic signatures (Spratt, D. E. et al. JCO 35, 1991-1998 (2017)). In JHMI, 72 (24%) men developed metastatic disease and in TCGA 82 (17.5%) developed disease recurrence.

### Statistical Analyses

All statistical tests were conducted using RStudio version 1.2.5019 (Boston, MA). Patient characteristics were compared using χ² to compare categorical variables and student T-test to compare age. Because stromal content and immune content in tumors positively correlate with each other and thus high levels of immune content may reflect high levels of stroma, when comparing immune content based on race, a logistic regression adjusting for stromal content with Black race as the outcome variable was performed. Individual immune cell type quantities were compared by race within the discovery cohort (JHMI) using the Wilcoxon rank-sum test, and similar comparisons were made in the first validation cohort (TCGA) with cell types that differed with a false-discovery rate (FDR) p<0.05. Parallel findings in TCGA were confirmed in the second validation cohort (DVA). Subsequent comparisons of continuous variables between two groups were assessed using the Wilcoxon rank-sum test unless otherwise noted. Spearman's correlation was used for correlation analyses. Continuous variables were categorized into equal quantiles for various comparisons. High vs low categorizations of continuous variables were divided by above vs below median unless otherwise noted. Kaplan-Meier analyses were used to estimate metastasis-free survival in the JHMI cohort and disease-free survival in TCGA. Log-rank analyses were used for univariable survival analyses while Cox proportional hazards models were used for multivariable analyses adjusting for clinical covariates. In multivariable Cox regressions. PSA was assessed as a log-transformed continuous variable. Grade group was assessed as a continuous variable per increase in 1 group from 1 (low grade) to 5 (high grade). T-stage was assessed as a categorical variable defined as organ confined as pT1-2 and NO and non-organ-confined as all others. Genomic risk score was defined as a transcriptomic-based validated score with higher values predicting increased risk of disease recurrence following local treatment for prostate cancer and was assessed as a continuous variable per 0.1 increase (range of possible scores: 0.0 to 1.0).

Prostate Tumors from Black Men have Increased Immune Content

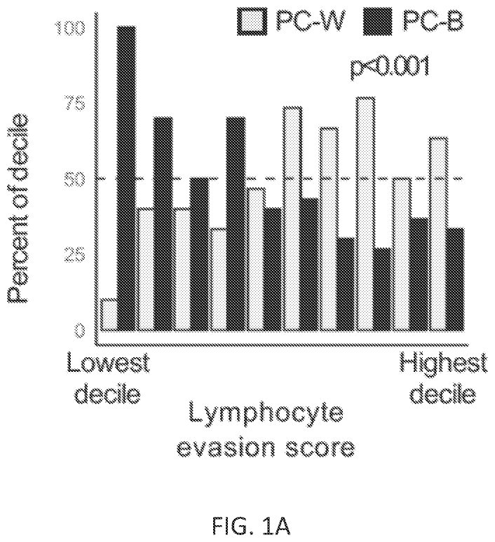

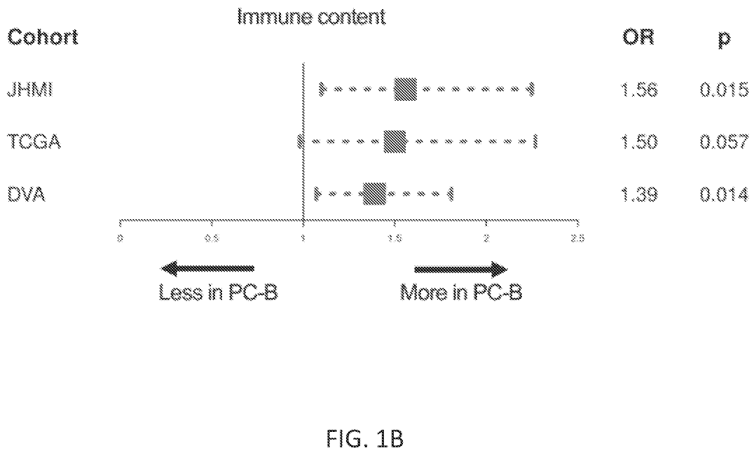

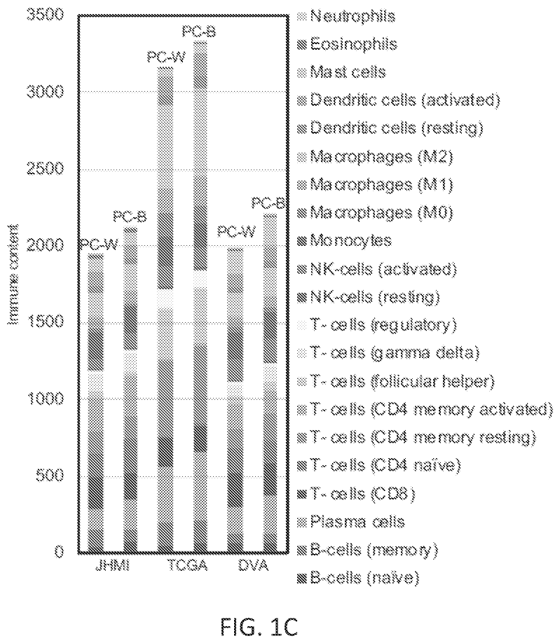

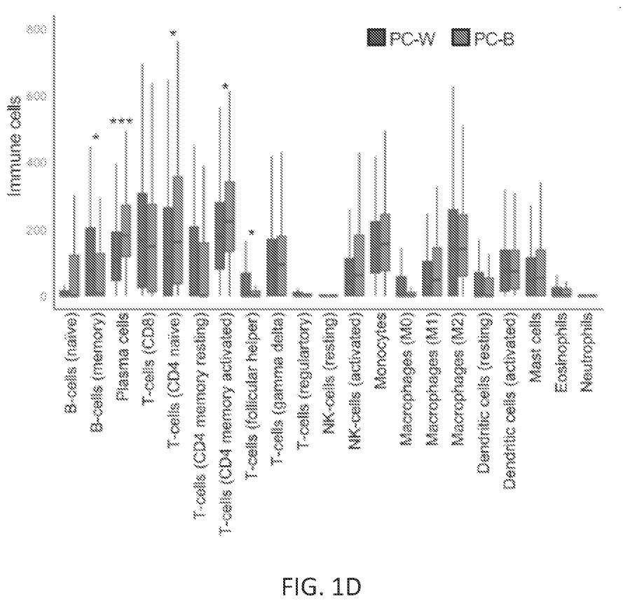

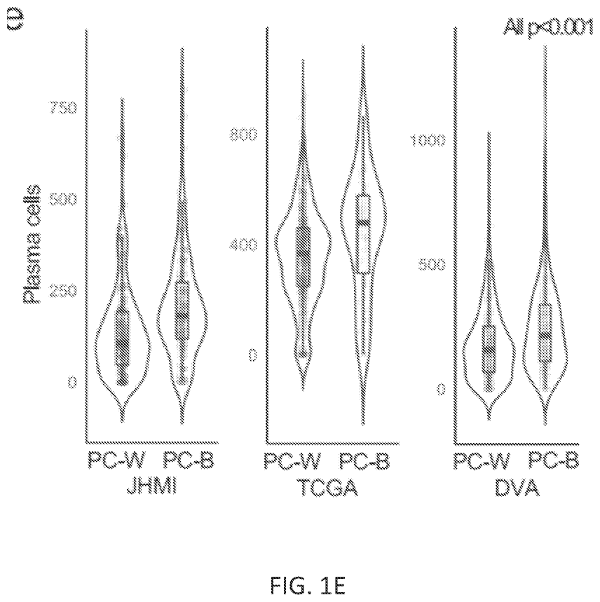

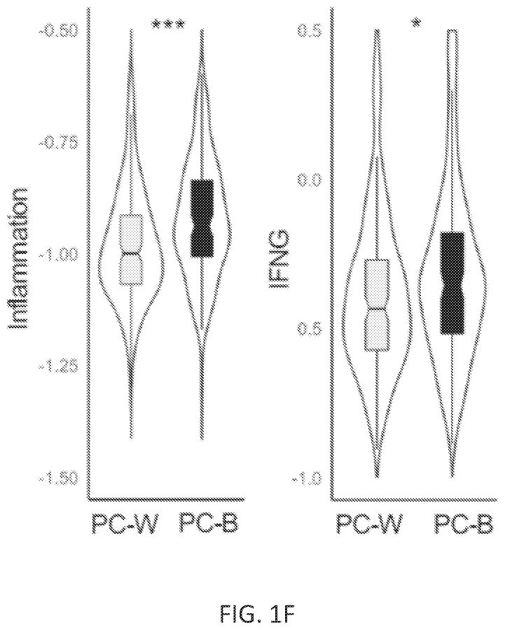

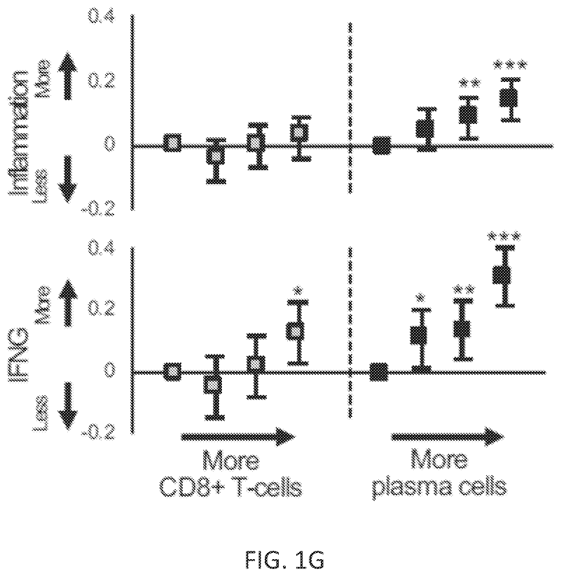

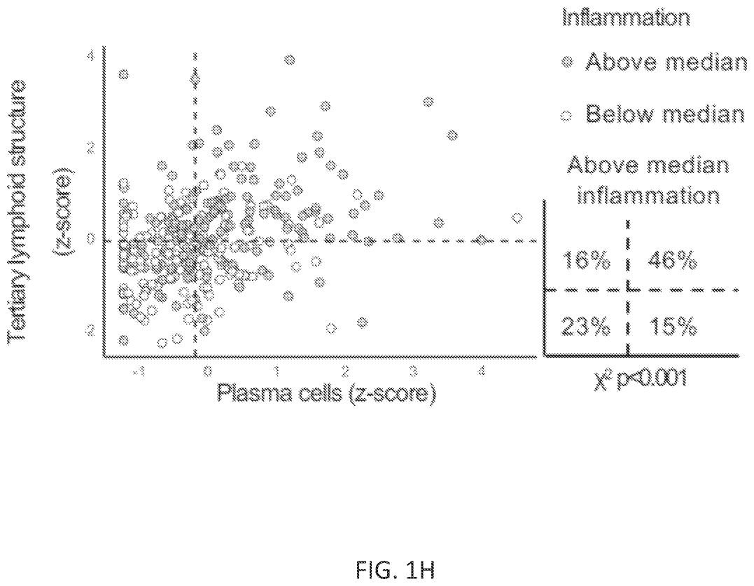

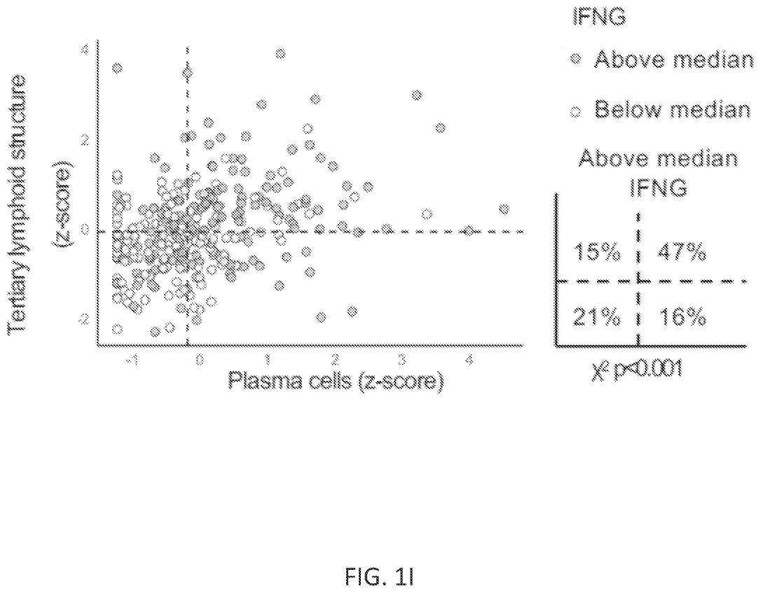

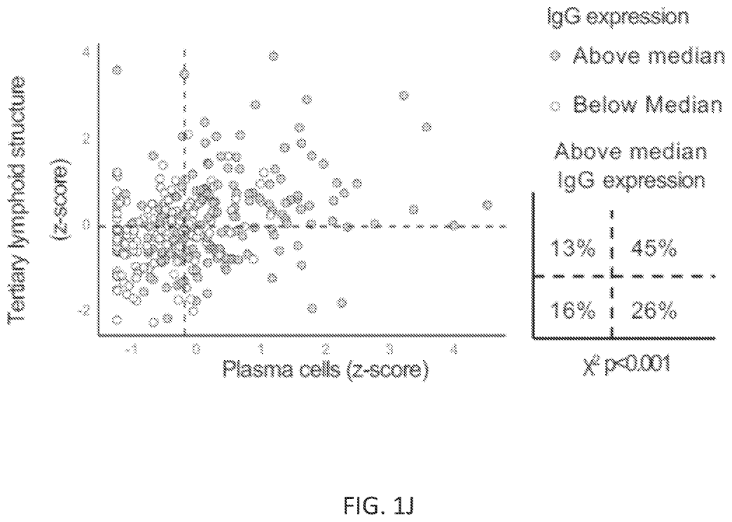

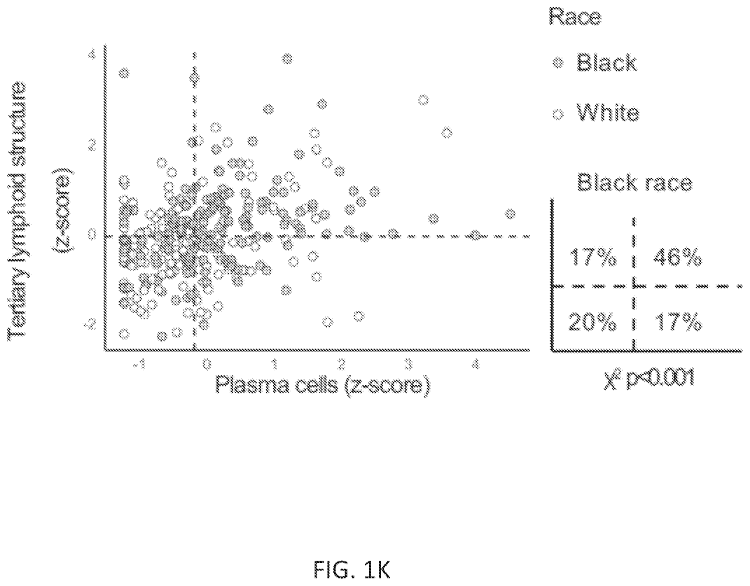

Utilizing a discovery cohort of intermediate- and high-grade primary PC from radical prostatectomy specimens grade- and stage-matched by race (Johns Hopkins Medical Institute [JHMI]; Black=150, [PC-B]; White=150, [PC-W]; Table 1) each tumor's ability to resist lymphocyte infiltration was assessed using an established expression-based signature of tumor lymphocyte evasion. Tumors with the lowest lymphocyte evasion scores were significantly enriched among PC-B suggesting PC-B tumors are more susceptible to lymphocyte infiltration into the TME (FIG. 1A). Correspondingly, PC-B had higher levels of immune content based on the ESTIMATE signature (FIG. 1B). Similar trends were noticed in two additional (validation) cohorts: 1) The Cancer Genome Atlas (TCGA; PC-B=58; PC-W=410; Odds ratio=1.50, p=0.057) and 2) a retrospective cohort of prostate tumors with whole transcriptomic data from radical prostatectomy at Durham Veterans Affairs Hospital (DVA; PC-B=302; PC-W=236; Odds ratio=1.39, p=0.014; Tables 2-3). After deconvoluting tumor invading lymphocytes by cell type (FIG. 1C), plasma cell content differed the greatest by race in JHMI and was the only cell type that differed by race in all cohorts (JHMI, TCGA, and DVA) with increased quantities in PC-B (FIGS. 1D-1E and Tables 5-7). Increases in B-lineage cells within PC-B was confirmed using two orthogonal methods including a DNA methylation-based tool (FIGS. 3A-B). To further validate these findings, we leveraged tumors from the previously described Kaur et al. cohort which showed PC-B possessed increased signals of B-lineage cells based on DNA methylation signatures (FIG. 3C) and both CD79a+ and CD138+ cell density (FIGS. 3D-3G).

TABLE 5
Plasma cell content quartiles by race in JHMI

| Plasma cell | PC-W, | PC-B, |
|---|---|---|
| content (Quartile) | n = 150 | n = 150 |
| 4 (Lowest) | 52 (34.7%) | 23 (15.3%) |
| 3 | 42 (28.0%) | 33 (22.0%) |
| 2 | 32 (21.3%) | 43 (28.7%) |
| 1 (Highest) | 24 (16.0%) | 51 (34.0%) |

Cochrane-Armitage test for trend, p < 0.001

TABLE 6
Plasma cell content quartiles by race in TCGA

| Plasma cell | PC-W, | PC-B, |
|---|---|---|
| content (Quartile) | n = 410 | n = 58 |
| 4 (Lowest) | 105 (25.6%) | 12 (20.7%) |
| 3 | 112 (27.3%) | 5 (8.6%) |
| 2 | 105 (25.6%) | 12 (20.7%) |
| 1 (Highest) | 88 (21.5%) | 29 (50.0%) |

Cochrane-Armitage test for trend, p < 0.001

TABLE 7
Plasma cell content quartiles by race in DVA

| Plasma cell | PC-W, | PC-B, |
|---|---|---|
| content (Quartile) | n = 236 | n = 302 |
| 4 (Lowest) | 75 (31.2%) | 60 (19.9%) |
| 3 | 62 (26.3%) | 72 (23.8%) |
| 2 | 57 (24.2%) | 77 (25.5%) |
| 1 (Highest) | 42 (17.8%) | 93 (30.8%) |

Cochrane-Armitage test for trend, p < 0.001

In a prospective trial of 19 men with advanced PC, tumors responsive to anti-CTLA-4 treatment were enriched with enhanced markers of inflammation and IFNG prior to treatment. Increased signatures of both inflammation and IFNG activity were noted in PC-B in JHMI (FIG. 1F) and increased IFNG in DVA and TCGA (FIG. 3H). Increasing quantities of plasma cells had a continuous positive association with both increasing inflammation and IFNG expression while CD8+ T-cells, the immune cells more commonly thought to mediate the most tumor immune activity, did not (FIG. 1G). In the setting of increased IFNG activity, IgG class-switch is augmented. With this in mind, we would expect tumors with high plasma cell content to also have increased markers of IgG expression. We found IgG expression correlated highly with plasma cell content (Spearman's correlation coefficient=0.65) suggesting tumors with high plasma cell content also have high IgG expression, potentially as a reflection of plasma cell antibody secretion (FIGS. 3I-3J). Additionally, plasma cell anti-tumor activity increases when they colocalize with other lymphocytes into organized cellular aggregates called tertiary lymphoid structures (TLSs). Thus, to further confirm the relationship between plasma cells and tumor immune activity. TLS activity was measured. We noted tumors with high TLS activity and plasma cell content had higher levels of inflammation, IFNG, and IgG expression, and were enriched within PC-B (FIGS. 1H-1K). Immunohistochemical assessment of T-cells, B-cells, and plasma cells in tumors with high TLS activity confirmed the presence of discrete lymphoid aggregates relative to tumors with low TLS activity (FIGS. 3K-3L). Thus, higher levels of plasma cells and TLSs define a subclass of tumors with higher immune activity which is more prevalent in tumors from Black men.

### Assessment of Potential Effect Modifiers

Several potential sources of effect modification for increased plasma cell content in PC-B were assessed. The association between PC-B and increased plasma cell content was independent of rearrangements in ERG and PTEN loss, two of the most common genomic alterations in PC which are less common in PC-B, (FIGS. 1N-1O and Table 8). In a prospective trial assessing the immune-based therapy sipuleucel-T, Black men with PC experienced longer overall survival compared to White men, in particular when baseline serum prostate-specific antigen (PSA) was low. Here, the association between PC-B and increased plasma cell content was independent of baseline PSA and age (Table 8). Finally, since the frequency of PC molecular subtypes differ by race, the interaction between subtype and race on plasma cell content in TCGA was assessed. We found the association between PC-B and increased plasma cell content was significantly enhanced among tumors classified as “other” non-subtypeable tumors (Estimate of interaction+159.58, 95% CI 16.62 to 301.55, p=0.028; Table 8). Accordingly, of the 16 PC-B classified as “other” non-subtypeable PC, 14 (87.5%) were found to have above median plasma cell content. Among these 14 tumors, no single gene was amplified or deep deleted more than three times and no gene was mutated more than twice. Since, “other” non-subtypeable PC comprises about 26% of tumors in TCGA and PC-B is more likely to not be classified as “other” non-subtypeable PC, these finding suggest a substantial portion of “other” non-subtypeable PC-B may be defined by their tumor immune microenvironment.

TABLE 8
Other subtype tumors from Black men in TCGA
with above-median plasma cell content

TCGA-EJ-8470
TCGA-G9-6378
TCGA-HC-7737
TCGA-HC-A4ZV
TCGA-KC-A4BL
TCGA-KK-A6ES
TCGA-KK-A7AV
TCGA-KK-A8IL
TCGA-V1-A8MF
TCGA-V1-A8MG
TCGA-V1-A8ML
TCGA-V1-A8MU
TCGA-V1-A8WL
TCGA-VP-A87E

### Plasma Cell Content Prognosticates Outcomes Following Surgery

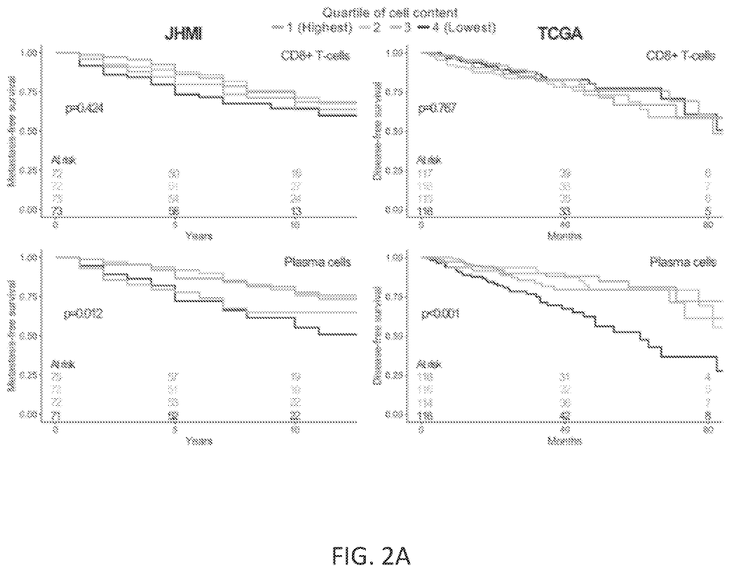

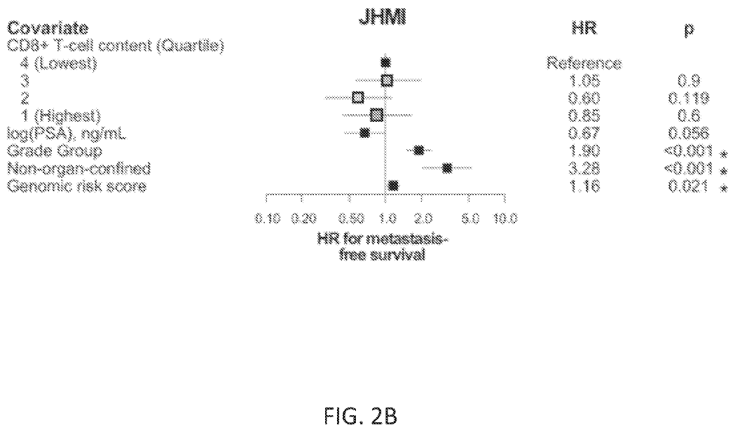

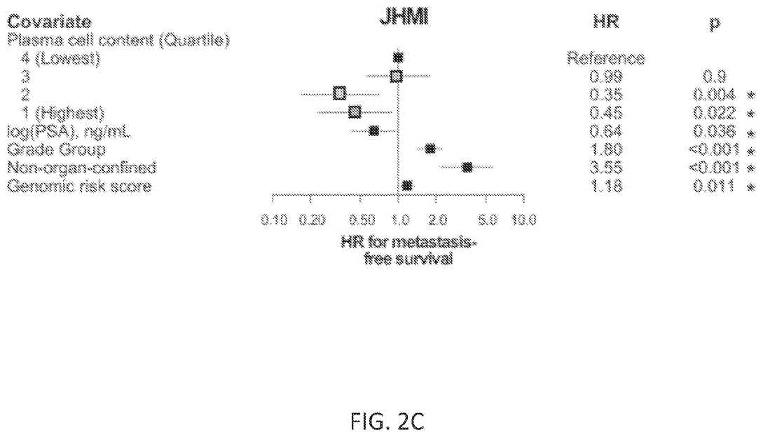

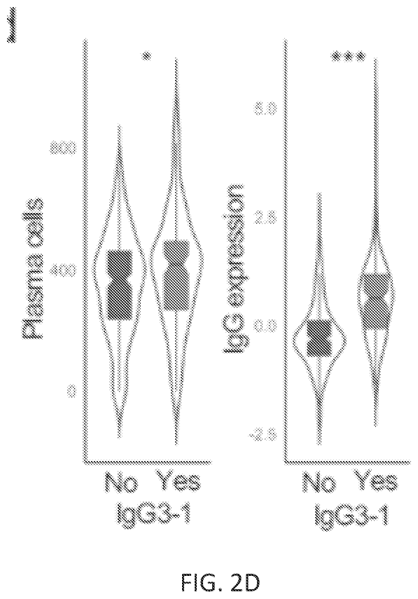

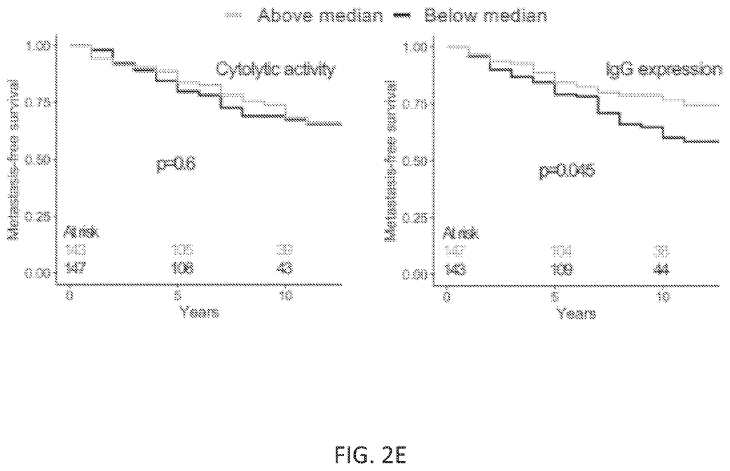

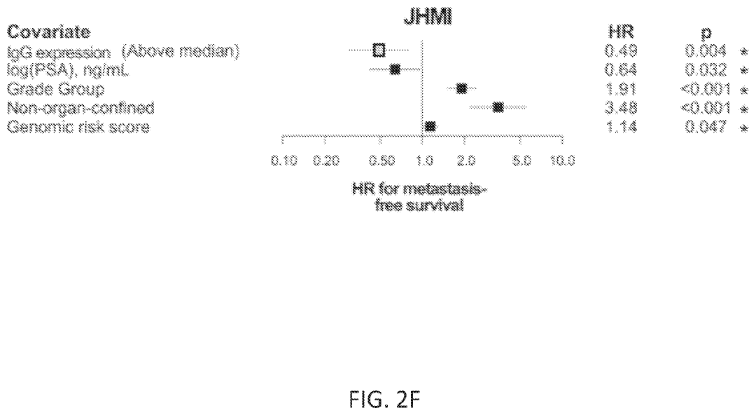

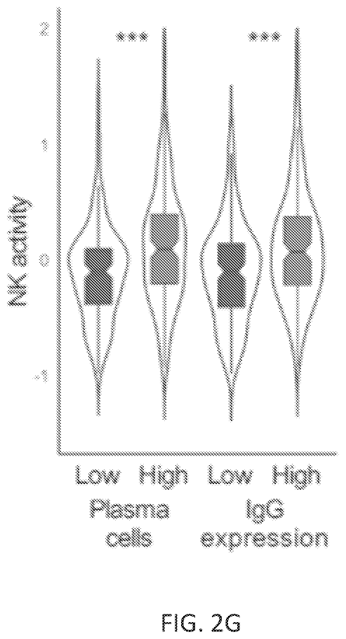

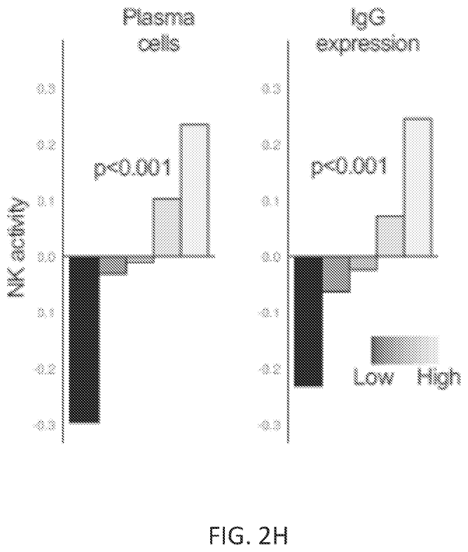

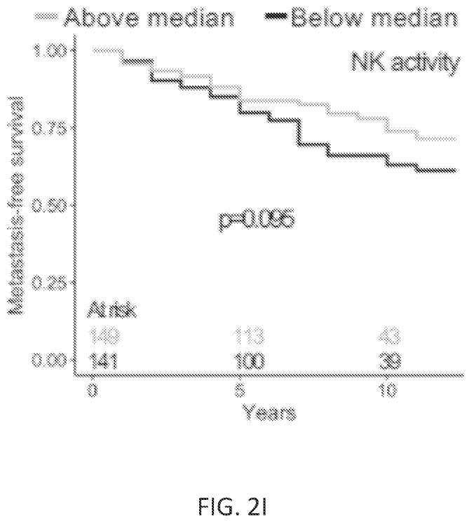

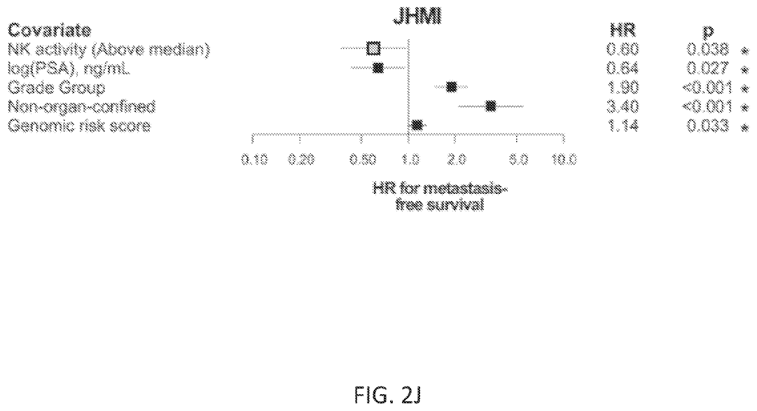

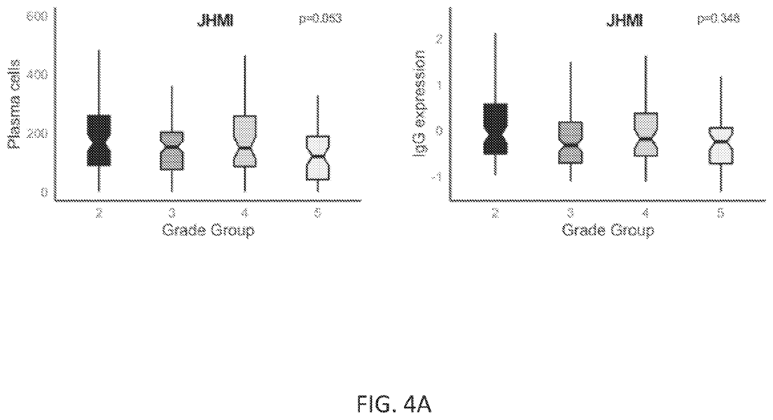

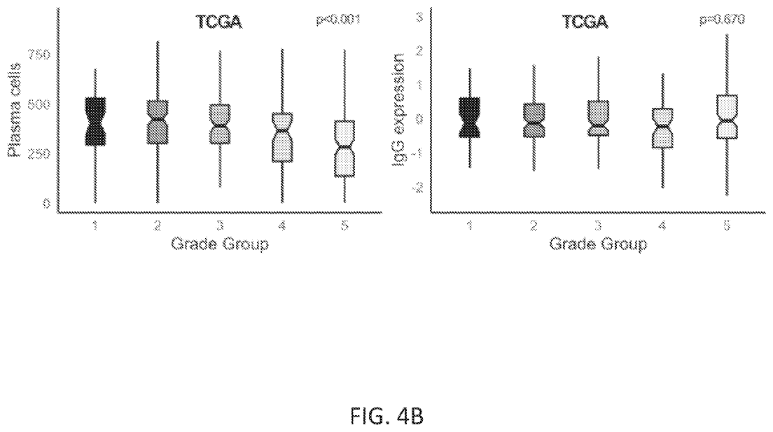

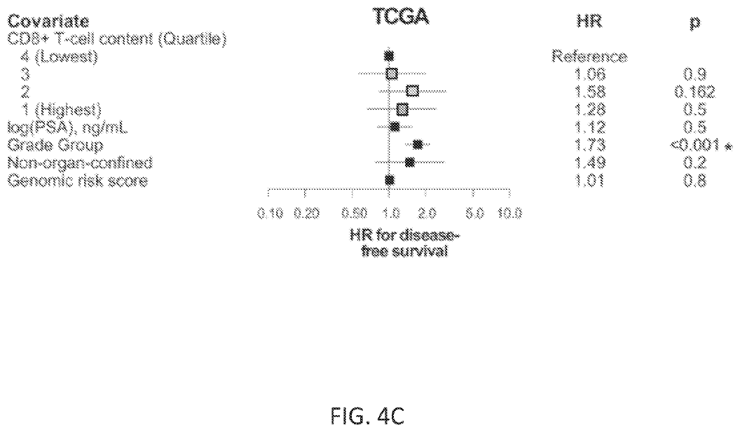

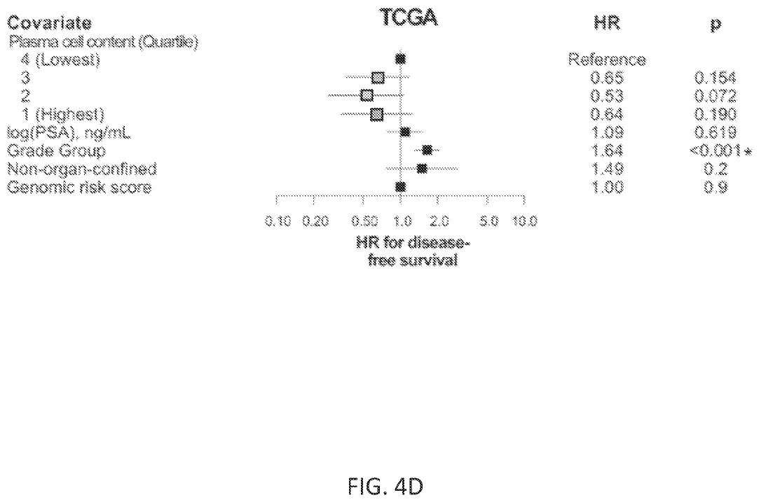

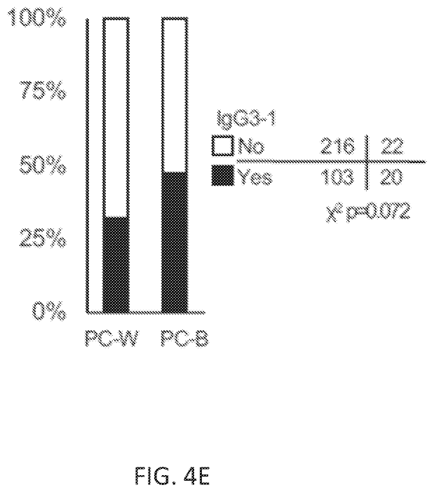

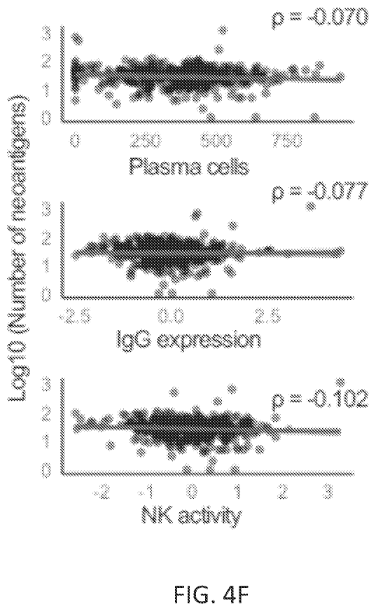

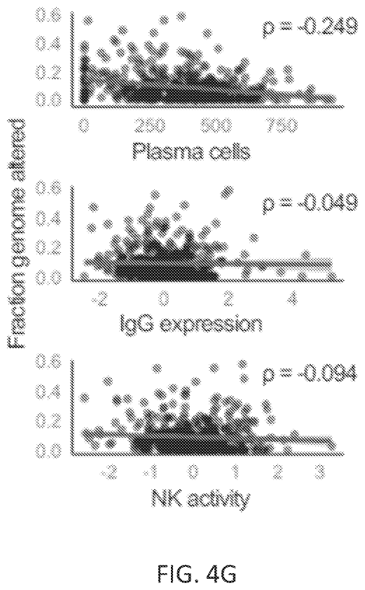

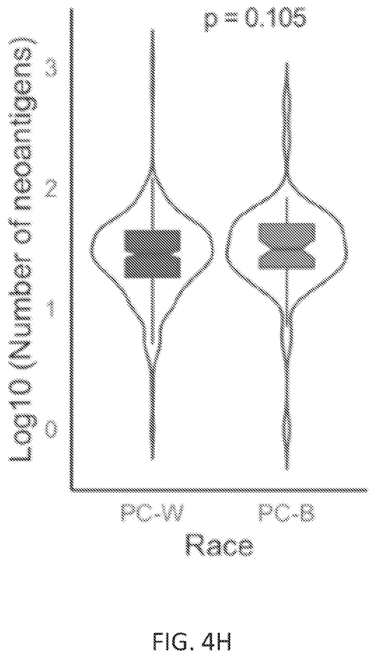

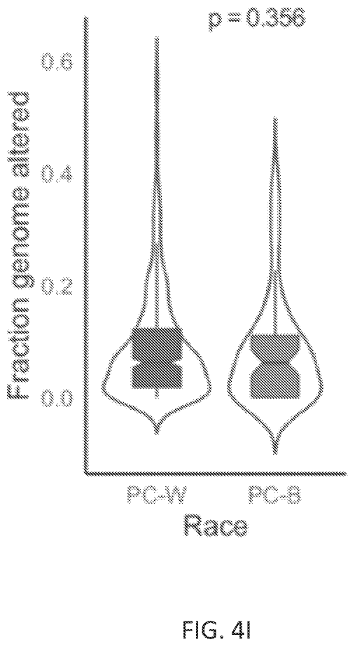

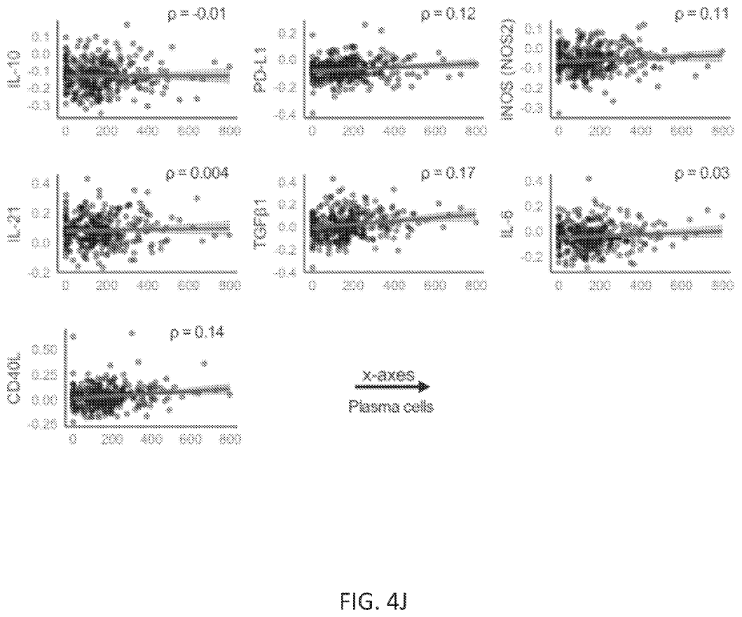

Developing metastatic disease following local definitive treatment is a strong surrogate for overall survival for men with localized prostate cancer. We found no association between metastasis-free and disease-free survival and CD8+ T-cell content. Conversely, increasing plasma cell content was associated with significantly prolonged metastasis-free and disease-free survival in JHMI and TCGA, respectively (FIG. 2A). In JHMI, plasma cell content and IgG expression did not tend to vary by grade, while in TCGA lower grade tumors tended to have higher plasma cell content (FIG. 4A-4B). In multivariable analysis including clinical covariates such as tumor grade, CD8+ T-cells remained non-significant while increased plasma cell content was independently associated with longer metastasis-free survival (FIG. 2B and FIGS. 4C-4D).

When B-cells encounter a foreign antigen, they undergo clonal expansion and immunoglobulin class-switch recombination. The most common class-switch recombination noted in multiple tumors types is IgG3 to 1 which is associated with cancer outcomes and can be measured by a signature originally described in an analysis by Hu et al. Nature Genetics 51, 560-567 (2019). In tumors from TCGA with any IgG3 to 1 class-switch recombination there were greater quantities of plasma cell content, more IgG expression, and a greater prevalence among PC-B (FIG. 2D and FIG. 4E). This observation suggests increasing plasma cell content and IgG expression may be reflective of B-cell antigen recognition within the TME. Accordingly, high IgG expression was associated with longer metastasis-free survival while high cytolytic activity, a measure of CD8+ T-cell activity was not (FIGS. 4E-4F). Tumor immune activity associated with plasma cell activity and IgG expression is typically mediated via antibody-dependent cellular cytotoxicity (ADCC) driven primarily by NK cell activation. Accordingly, we noted increased NK activity in tumors with high levels of plasma cells and IgG expression (FIGS. 2G-2H). Plasma cell content, IgG expression, and NK activity were all independent of tumor neoantigen burden and fraction genome altered in TCGA consistent with previous work on tumor plasma cell content and TLS in melanoma and sarcoma (FIGS. 4F-4G). Similarly, neoantigen burden and fraction genome altered did not correlate with race (FIGS. 4H-4I). In the JHMI cohort, high NK activity was associated with longer duration to metastatic disease following radical prostatectomy (FIGS. 2I-2J).

As noted herein, plasma cell content was positively associated with IFNG in primary PC (FIG. 1G). Conversely, plasma cell content was not strongly correlated with the expression of several relevant inflammatory factors that induce IgA class switch (FIG. 4J). Thus, discrepancies in the TME inflammatory cytokine milieu leading to differential Ig isotype switching may account for the heterogeneous observed associations between plasma cell and PC outcomes.

These results showed that increases in plasma cell infiltrate and segmented markers of NK cell activity and IgG expression are associated with improved recurrence-free survival following surgery for prostate cancer. These results suggested that the methods of the disclosure would be useful for identifying prostate cancer patients that would likely respond to immunotherapy. These results further suggested that the methods and signatures of the present disclosure would be useful for diagnosing, prognosing, determining the progression of cancer, or predicting benefit from immunotherapy in a subject having prostate cancer.

### Example 2: Validation of an Expression-Based Marker of Intratumoral Plasma Cell Content with Immunohistochemistry to Identify Prostate Tumors that are Likely to Respond to Immunotherapy

An expression-based marker of intratumoral plasma cell content to identify tumors that will likely be responsive to immunotherapy was validated as follows. Study cohorts included consisted of three datasets of molecular and clinical data: two from tumors sampled from radical prostatectomy (RP) specimens and one from biopsies of men with metastatic castration-resistant PCa (mCRPC). The first RP cohort from Johns Hopkins Medical Institute (n=498) was comprised of patients who underwent RP between 1995 and 2010 with no adjuvant treatments until end of follow-up or development of metastatic disease (see Table 9). The second RP cohort from the Decipher Genomic Resource Information Database (GRID; NCT02609269) was comprised of tumors collected prospectively from the clinical use of the Decipher RP test from December 2015 through September 2017. In this group, the study focused on those with high grade (grade groups 4 or 5) tumors given the inherent interest in adjuvant treatments for this group of patients who are at high risk of recurrence following definitive treatment (n=785) (see Table 10). Detailed information on these two cohorts including expression profiling and clinical outcomes has been previously described (Weiner et al. Prostate Cancer Prostatic Dis, 2021).

TABLE 9
Johns Hopkins Medical Institute cohort

| Characteristic | Median/n (IQR/%) | |
|---|---|---|
| Total | 498 | (100) |
| Age (years) | 59 | (55-72) |

Grade group

| | | |
|---|---|---|
| 1 | 30 | (6) |
| 2 | 174 | (34.9) |
| 3 | 123 | (24.7) |
| 4 | 59 | (11.8) |
| 5 | 112 | (22.5) |
| PSA (ng/mL) | 8.0 | (5.48-12.9) |
| Unknown | 2 | (0.4) |

Organ confined

| | | |
|---|---|---|
| Yes | 342 | (68.7) |
| No | 150 | (30.1) |
| Unknown | 6 | (1.2) |

Metastatic recurrence

| | | |
|---|---|---|
| No | 356 | (71.5) |
| Yes | 142 | (28.5) |
| Follow-up (years) | 8 | (5-12) |

Abbreviations: PSA, prostate-specific antigen; IQR, interquartile range

TABLE 10
Stand Up to Cancer 2019 Cohort

| Characteristic | Median/n (IQR/%) | |
|---|---|---|
| Total | 101 | (100) |
| Age (years) | 60.6 | (55.7-65.6) |

Treatment exposure

| | | |
|---|---|---|
| Yes taxane, Yes ARSi | 25 | (24.8) |
| Yes taxane, No ARSi | 10 | (9.9) |
| No taxane, Yes ARSi | 25 | (24.8) |
| No taxane, No ARSi | 41 | (40.6) |

Abbreviations: IQR, interquartile range; ARSi, androgen receptor signaling inhibitors

The mCRPC cohort was derived from the publicly available information describing the 2019 iteration of the Stand Up To Cancer (SU2C2019) dataset. (Abida et al. PNAS 116:11428-11436, 2019.) This study focused on patients in SU2C2019 with known information on previous exposures to taxane chemotherapy or androgen receptor signaling inhibitors (ARSi; abiraterone or enzalutamide) and only those with biopsies from bone to minimize the heterogeneity in tumor immune content related to biopsy site (n=101) (see Table 11). All the data for the SU2C2019 cohort was downloaded from eBioPortal. (Cerami et al. Cancer Discov 2:401-404, 2012 and Gao et al. Sci Signal 6:pl1-pl1, 2013.) Expression data from SU2C2019 was quantile matched to the microarray platform used for the GRID cohort.

TABLE 11
GRID cohort

| Characteristic | Median/n (IQR/%) | |
|---|---|---|
| Total | 785 | (100) |
| Age (years) | 66.5 | (62.0-70.5) |

Grade group

| | | |
|---|---|---|
| 4 | 334 | (42.5) |
| 5 | 451 | (57.5) |
| PSA (ng/mL) | 7.0 | (5.19-11.5) |

Organ confined

| | | |
|---|---|---|
| Yes | 187 | (23.8) |
| No | 567 | (72.2) |
| Unknown | 31 | (3.9) |

Abbreviations: GRID, Genomic Resource Information Database; PSA, prostate-specific antigen; IQR, interquartile range

### Immune Cell Content

Tumor immune content was calculated using the ESTIMATE R package version 2.0.0.23 Tumor-invading lymphocytes were deconvoluted using the MySort tool (default version) implemented in an R function. The MySort tool produces the proportions of each immune cell type in each tumor. To apply these proportions to the immune content from ESTIMATE and avoid negative values, total immune content was the final immune score from ESTIMATE plus the constant of the minimum score in each cohort plus one.

### CD138+ Densities

Immunohistochemistry for CD138/syndecan-1, a marker of plasma cells, within the JHMI cohort (n=113) was conducted using mouse monoclonal antibodies (B-A38, Ventana/Roche, pre-dilute) on the Ventana Benchmark platform in a CLIA-accredited laboratory. As previously described, we performed a manual quantification of CD138+ cell density blinded to clinical and patient characteristics. Individual cells positive for CD138 were counted in each 0.6 mm diameter tissue core of tumors (average of four spots sampled per case), normalized by the total mm² of tissue analyzed for each core calculated in QuPath version 0.1.2, taking the mean across the four tumor cores for each patient. The manual quantification was done due to the observed patchy expression of CD138 in benign prostatic epithelium.

### Expression-Based Signatures

Two signatures predictive of responses to immunotherapy were used. “Immunotherapy score 1” (Ayers et al. J Clin Invest 127:2930-2940, 2017) and “Immunotherapy score 2” (Cristescu et al. Science 362:eaar3593, 2018) were both developed and validated to predict clinical response to anti-programmed cell death protein 1 (anti-PD-1) therapies. The androgen receptor (AR) activity signature was based on the weighted expression of nine canonical androgen receptor transcriptional target genes (KLK3, KLK2, FKBP5, STEAP1, STEAP2, PPAP2A, RAB3B, ACSL3, and NKX3-1) validated to measure tumors androgen receptor output and response to androgen deprivation therapy. (Spratt et al. Clin Cancer Res 25:6721-6730, 2019.) Radiation therapy response score was based on the Post-operative radiotherapy outcomes score (PORTOS). (Zhao et al. Lancet Oncol 17:1612-1620, 2016.) Genomic risk classifier was defined as an expression-based score validated specifically for PCa to predict risk of disease recurrence following local treatment with higher scores (0.0 to 1.0) predicting an increased risk. (Spratt et al. JCO 35:1991-1998, 2017.) Tumor immunophenoscores and their major and minor parameters were calculated as previously described. (Charoentong et al. Cell Rep 18:248-262, 2017.)

### Master Regulator Analysis

Master regulator (MR) analyses were performed using the MARINa algorithm (Carro et al. Nature 463:318-325, 2010 and Lefebvre et al. Mol Syst Biol 6:377, 2010) on the plasma cell signature and VIPER algorithm (Alvarez et al. Nat Genet 48:838-847, 2016) on single-patient level which are available for download at http://califano.c2b2.columbia.edu/software/ and as viper R package from Bioconductor, using the same parameters as described previously. (Alvarez et al. Nat Genet 48:838-847, 2016 and Aytes et al. Cancer Cell 25:638-651, 2014.) MARINa and VIPER analyses were applied in GRID (n=784) and JHMI (n=498) cohorts, separately. Note that one sample (DPR011934.CEL) from GRID was removed from the analysis as an outlier identified from principal components analysis (PCA) using the prcomp function in R.

For MARINa analysis, the plasma cell signature was used to define as a ranked list of genes based on their differential expression (two-sides Welch t-test) between plasma cell-high (content greater than mean+1 standard deviation) and plasma cell-low tumors in the GRID cohort and utilized to query prostate-cancer-specific regulatory network (interactome), as reconstructed in Aytes et al. (Aytes et al. Cancer Cell 25:638-651, 2014.) VIPER analysis was applied to GRID and JHMI datasets separately, where in each dataset, patient samples were scaled (z-scored) on a gene-level prior to conducting the analyses, which allowed applying VIPER algorithm on a single patient level. MR activity levels expressed as normalized enrichment scores from VIPER analysis on the JHMI cohort were utilized for multivariable Cox proportional hazards survival analysis, with metastatic recurrence as the endpoint. In survival analyses, high MR activity was defined as any tumor above the upper quartile of activity within the JHMI cohort.

### Statistics

All statistical tests were conducted using R Studio version 1.2.5019 (Boston, MA) and only two-sided p-values were generated. Following MR analysis and calculation of MR activity level (see above), each tumor in JHMI was divided by low versus high activity level based on the upper quartile of activity level for each regulator. Multivariable Cox proportional hazards regression analyses were performed to calculate the hazard ratio (HR) and 95% confidence interval (CI) for high versus low activity for each master regulator adjusting for grade group, log-transformed serum prostate-specific antigen (PSA), and organ confined versus not organ confined disease.

### Results

### Expression-Based Signature for Plasma Cells Content Validated by Immunohistochemistry

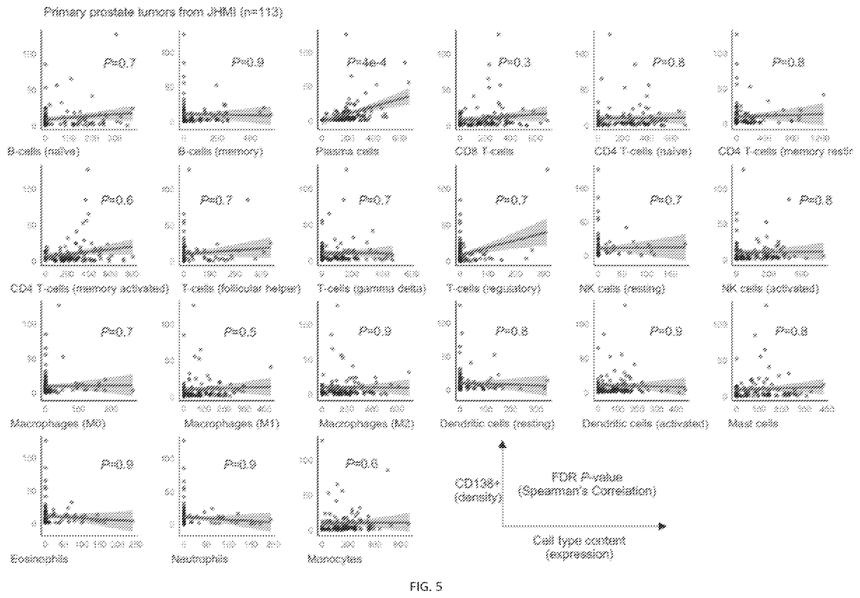

CD138+ density, an immune cell marker specific to plasma cells, (O'Connell et al. Am J Clin Pathol 121:254-263, 2004) on immunohistochemistry was quantified in 113 tumors from the JHMI cohort. As shown in Table 12 below, Spearman's correlation p and false discovery rate (FDR)-adjusted P-values were calculated between CD138+ density and each immune cell type content deconvoluted (FIG. 5). Plasma cell content was the only cell type significantly associated with CD138+ density validating the expression-based signature as a marker of plasma cell content in PCa.

TABLE 12
Spearman's correlation between immune
cell content and CD138+ density

| Cell type | Rho | P-value | FDR P-value |
|---|---|---|---|
| B-cells (naïve) | 0.09171936 | 0.340599 | 0.68233047 |
| B-cells (memory) | −0.0282067 | 0.76989409 | 0.95104564 |
| B cells (plasma) | 0.39344984 | 2.12E−05 | 0.00044451 |
| CD8 T-cells | 0.20328615 | 0.03316984 | 0.34828327 |
| CD4 T-cells (naïve) | 0.07578239 | 0.4313607 | 0.75430672 |
| CD4 T-cells | 0.04472944 | 0.64264804 | 0.84347555 |
| (memory resting) | | | |
| CD4 T-cells | 0.13509441 | 0.15938111 | 0.63655343 |
| (memory activated) | | | |
| T-cells (follicular | 0.08903622 | 0.35497055 | 0.68233047 |
| helper) | | | |
| T-cells (gamma delta) | −0.1079238 | 0.26174952 | 0.68233047 |
| T-cells (regulatory) | −0.0885873 | 0.3574112 | 0.68233047 |
| NK cells (resting) | 0.13559405 | 0.15782961 | 0.63655343 |
| NK cells (activated) | 0.0581688 | 0.54609518 | 0.76453325 |
| Macrophages (M0) | 0.09879677 | 0.30448302 | 0.68233047 |
| Macrophages (M1) | 0.17569311 | 0.06636601 | 0.46456204 |
| Macrophages (M2) | 0.00343922 | 0.97155437 | 0.98388982 |
| Dendritic cells | 0.06644824 | 0.49037237 | 0.75430672 |
| (resting) | | | |
| Dendritic cells | 0.00329058 | 0.9727833 | 0.98388982 |
| (activated) | | | |
| Mast cells | 0.06455072 | 0.50287115 | 0.75430672 |
| Eosinophils | −0.0170708 | 0.85950198 | 0.98388982 |
| Neutrophils | −0.0019475 | 0.98388982 | 0.98388982 |
| Monocytes | 0.12822705 | 0.18187241 | 0.63655343 |

Abbreviation: FDR, false discovery rate.

Plasma cell content decreases following systemic therapies and defines a biologically distinct subgroup of high risk tumors

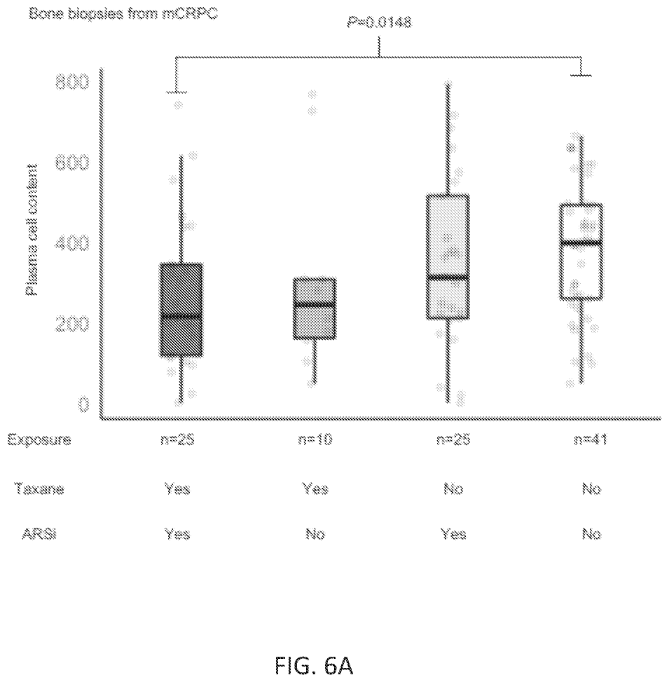

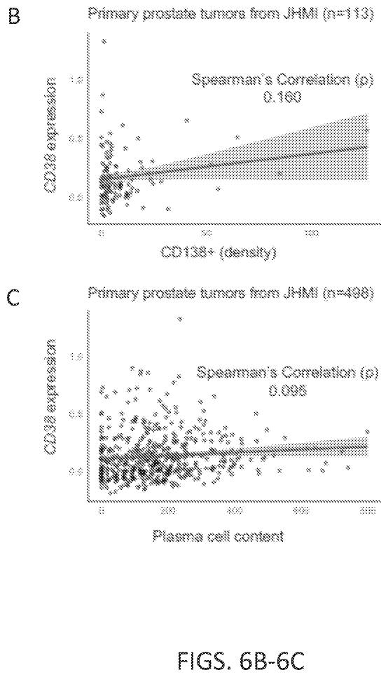

In advanced PCa, tumors from patients who received fewer systemic treatments had more pre-treatment tumor immune content and favorable responses to anti-CTLA4 immunotherapy. (Subudhi et al. Science Translational Medicine 12:eaaz3577, 2020.) In bone biopsies of mCRPC in the SU2C2019 cohort, tumors from patients who were not previously exposed to taxane chemotherapy or androgen receptor signaling inhibitors (abiraterone or enzalutamide) possessed higher plasma cell content than those with previous treatment exposures (FIG. 6A) (see Table 13). Prior work in advanced PCa have implicated B-cells in immunosuppression and tumor immune evasion. (Shalapour et al. Nature 521:94-98, 2015 and Guo et al. European Urology 79:736-746, 2021.) We previously showed increased intratumoral plasma cell content was associated with increased immune activity within primary Pca. (Weiner et al. Nat Commun 12:935, 2021.) Discrepancies in the tumor microenvironments inflammatory cytokine milieu which induce differential antibody isotype switch could account for the heterogeneous (pro-tumorogenic versus anti-tumorogenic) roles of plasma cells in Pca. (Weiner et al. Nat Commun 12:935, 2021.) Guo et al. showed increasing expression of CD38 (a marker of many immune-cells including mature, plasma B-cells) was associated with immunosuppressive pathways and worse oncologic outcomes for patients with CRPC. (Guo et al. European Urology 79:736-746, 2021.) In primary Pca, CD38 expression only weakly correlated with CD138+ density and deconvoluted plasma cell content (FIG. 6B-6C). Together, these findings in advanced PCa suggest systemic therapies or tumor progression coincide with a relative resistance to anti-tumor immune responses and upregulation of immunosuppressive pathways.

TABLE 13
Linear regression between previous treatment exposure
and plasma cell content in SU2C2019 (n = 101)

| | | |
|---|---|---|
| Treatment exposure | Plasma cell content (95% CI) | P |

| | |
|---|---|
| Yes taxane, Yes ARSi | Reference |

| | | | |
|---|---|---|---|
| Yes taxane, No ARSi | +34.3 | (−109.1 to +177.7) | 0.6 |
| No taxane, Yes ARSi | +74.4 | (−34.0 to +182.8) | 0.2 |
| No taxane, No ARSi | +105.3 | (+8.0 to +202.5) | 0.034 |

Abbreviations: CI, confidence interval; ARSi, androgen receptor signaling inhibitors

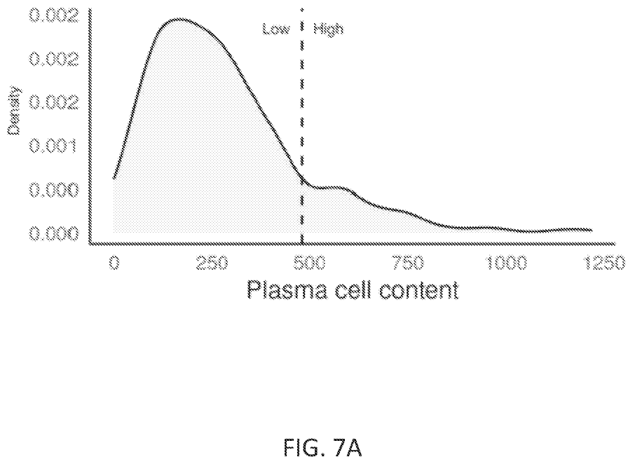

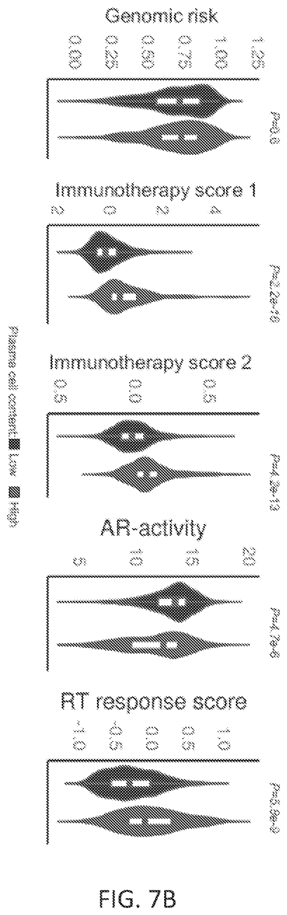

In that context, we sought to characterize plasma cell content in early stage, treatment naïve, high grade PCa. This disease group has a significant potential for progression to lethal disease following definitive treatment and may benefit from neoadjuvant treatments. (Tewari et al. Journal of Urology 177:911-915, 2007.) Within the GRID cohort (n=785), high grade tumors with high plasma cell content possessed similar genomic risk scores based on a validated expression-based score suggesting similar phenotype in terms of predicted aggressiveness (FIG. 7A-7B). (Spratt et al. JCO 35:1991-1998, 2017) Patients with high genomic risk scores might benefit from adjuvant or early salvage treatments following radical prostatectomy. Notably, despite similar genomic risk scores, tumors with high plasma cell content had predicted increased responses to immunotherapy and radiation therapy, and lower androgen receptor output suggesting differential benefits from certain treatments (FIG. 7B). These findings suggest despite having similarly aggressive phenotypes compared to primary high grade tumors with low plasma cell content, tumors with high plasma cell content might benefit more from immune-based treatments and less from androgen deprivation therapy following initial definitive treatment.

Immune-Based Pathways are Upregulated in Tumors with High Plasma Cell Content

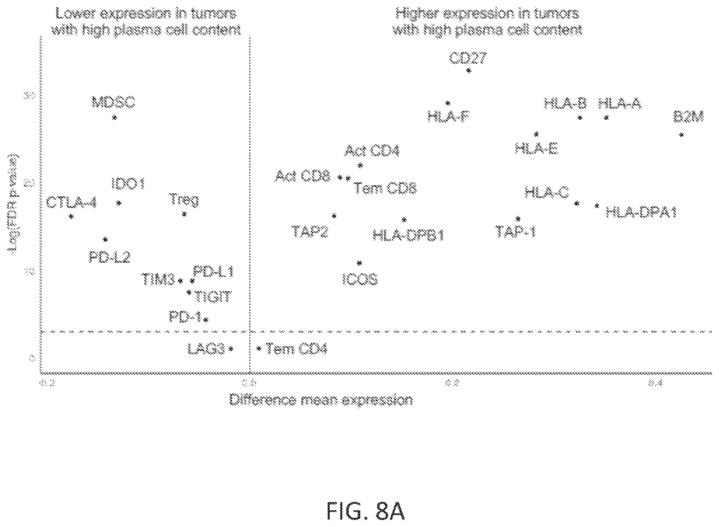

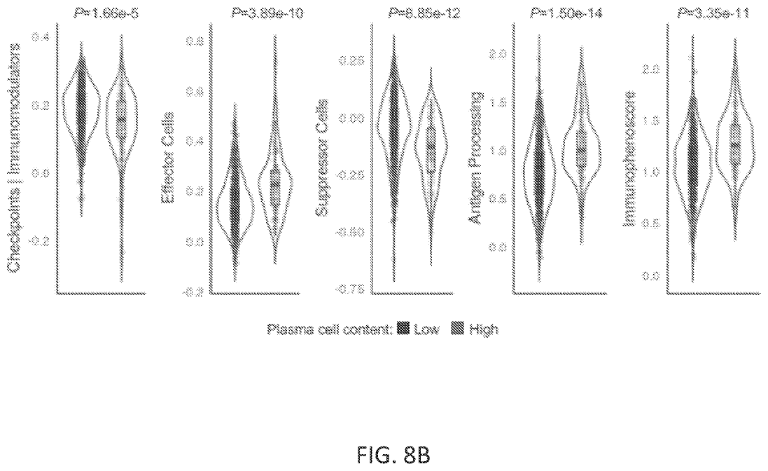

Using the validated components of the immunophenoscore, (Charoentong et al. Cell Rep 18:248-262, 2017) high grade tumors within the GRID cohort were noted to be associated with immune pathways associated with increased immune activity (e.g. increased HLA expression; FIG. 8A). Accordingly, tumors with high plasma cell content were associated with increased pathways in effector cells and antigen presentation, decreased pathways in checkpoint/immunomodulators and suppressor cells, and overall high immunophenoscores suggesting predicted increased response to immunotherapy (FIG. 8B).

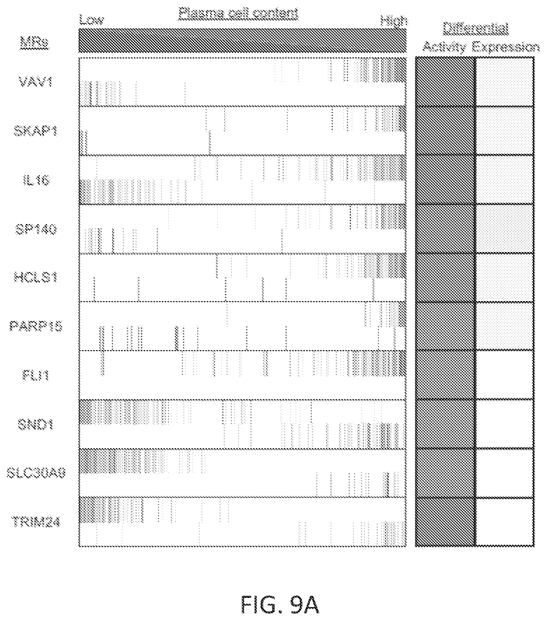

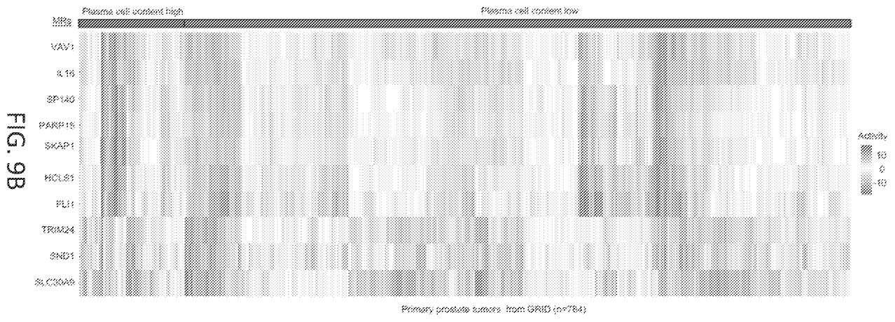

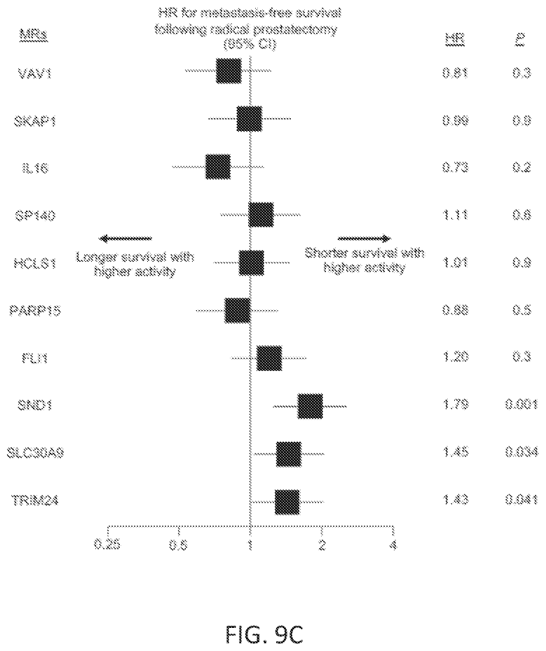

We then sought to identify immune-related transcriptional regulatory programs to explain the increased immune pathways in tumors with high plasma cell content. For this, we categorized tumors in the GRID cohort as plasma cell-high and plasma cell-low. This signature was then subjected to MR MARINa analysis, which utilized prostate-cancer specific transcriptional regulatory network (interactome) and identified MRs based on enrichment (differential expression) of their transcriptional targets. From the top 10 MRs, this analysis identified seven MRs with increased activity in plasma cell-high tumors and three with decreased activity (FIG. 9A). To confirm the transcriptional activity patterns across patients in plasma cell-high and plasma cell-low patient groups, we performed the single-patient VIPER analysis in the GRID cohort. While there was some heterogeneity in this arrangement of differential activity among tumors with high plasma cell content, most of these tumors did express this pattern (FIG. 9B).

The MR most over-activated in plasma cell-high tumors was VAV1 (Vav Guanine Nucleotide Exchange Factor 1) which has been implicated in B-cell maturation. (Tedford et al. Nat Immunol 2:548-555, 2001.) The MR most over-activated in plasma cell-low tumors was TRIM24 (tripartite motif-containing protein 24) which has previously been shown to augment androgen receptor output in SPOP mutant PCa. (Groner et al. Cancer Cell 29:846-858, 2016.) To explore association of the activity levels of the 10 MRs with tumor aggressiveness, we estimated activity levels for these 10 MRs on a single-patient level in the JHMI cohort using VIPER algorithm and utilized them as inputs into Cox proportional hazards models with metastatic recurrence as the end-point. In multivariable Cox proportional hazards regressions adjusting for tumor grade and stage, and serum PSA, the three MRs with increased activity in tumors with low plasma cell content, including TRIM24, were each significantly associated with shorter time to metastatic recurrence following surgery (FIG. 9C).

These results indicated that intratumoral plasma cell content defines primary PCa with distinct treatment vulnerabilities, upregulated immunogenic pathways, and differentially activated MRs. Together these results suggested that measuring intratumoral plasma is useful for patient selection for immune-based treatments.

In this study, we validated an expression-based marker of intratumoral plasma cell content with immunohistochemistry. In prostatectomy specimens, CD138+ density correlated significantly with the expression-based marker of plasma cells and no other cell types suggesting the marker was specific for plasma cells in PCa. Importantly, in mCRPC we showed tumors from patients who received fewer systemic treatments expressed higher plasma cell content. In this study, overexpression of each MR activated in plasma cell-low tumors was associated with shorter time to metastatic disease following surgery. In high grade primary PCa, tumors with high plasma cell content were associated with increased predicted response to immunotherapy and decreased response to androgen-deprivation therapy.

These results showed that an expression-based marker of intratumoral plasma cell content with immunohistochemistry is useful for predicting benefit from immunotherapy for a subject who has prostate cancer. These results further showed that the methods of the disclosure are useful for predicting benefit from immunotherapy for a subject who has prostate cancer. These results also showed that the methods of the disclosure would be useful for determining a treatment for a patient who has prostate cancer. These results suggested that the methods of the disclosure would be useful for identifying prostate cancer patients that would likely respond to immunotherapy. These results further suggested that the methods and signatures of the present disclosure would be useful for diagnosing, prognosing, determining the progression of cancer, or predicting benefit from immunotherapy in a subject having prostate cancer.

While embodiments of the disclosure have been illustrated and described, it will be appreciated that various changes can be made therein without departing from the spirit and scope of the disclosure.

1-22. (canceled)

23. A method for treating a patient for prostate cancer, the method comprising:
a) assaying or having assayed a level of immune content in a biological sample comprising immune cells and/or prostate cancer cells from the patient with prostate cancer;
b) determining or having determined whether or not the patient is likely to be responsive to immunotherapy based on the level of immune content in the biological sample; and
c) administering immunotherapy to the patient if the patient is identified as likely to be responsive to immunotherapy, or administering a cancer treatment other than immunotherapy to the patient if the patient is not identified as likely to be responsive to immunotherapy.

24. (canceled)

25. The method of claim 23, wherein the immunotherapy is cellular immunotherapy, antibody immunotherapy, and/or cytokine immunotherapy.

26. The method of claim 23, wherein the immunotherapy is sipuleucel-T.

27. The method of claim 23, further comprising performing surgery, radiation therapy, chemotherapy, hormonal therapy, biologic therapy, or any combination thereof.

28. A kit for predicting response of a subject to immunotherapy, the kit comprising agents for measuring levels of immune content in a biological sample comprising immune cells and/or prostate cancer cells from a subject having prostate cancer.

29. (canceled)

30. (canceled)

31. (canceled)

32. (canceled)

33. A method of diagnosing, prognosing, determining the progression of cancer, or predicting benefit from immunotherapy in a subject with prostate cancer, comprising:
a) assaying a level of immune content in a biological sample comprising immune cells and/or prostate cancer cells from the subject; and
b) diagnosing, prognosing, determining the progression of cancer, or predicting benefit from immunotherapy in the subject based on the level of immune content in the biological sample.

34. The method of claim 23, wherein a higher level of immune content compared to a control reference value indicates that the patient will likely respond to immunotherapy.

35. The method of claim 23, wherein the immune content is plasma cells, tertiary lymphoid structure activity, IgG expression, interferon gamma (IFNG) signaling, and/or natural killer (NK) cell activity.

36. The method of claim 35, wherein the plasma cell levels are increased in the sample, wherein the tertiary lymphoid structure activity level is increased in the sample, wherein the IgG expression level is increased in the sample, wherein the IFNG signaling level is increased in the sample, wherein the NK cell activity level is increased in the sample, and/or wherein the CD138/syndecan-1 level is increased in the sample.

37-41. (canceled)

42. The method of claim 23, wherein assaying or having assayed the level of immune content comprises assaying or having assayed the expression level for any one or more of the following markers KLK3, KLK2, FKBP5, STEAP1, STEAP2, PPAP2A, RAB3B, ACSL3, and NKX3-1.

43. The method of claim 23, wherein assaying or having assayed the level of immune content comprises assaying or having assayed the expression level of any one or more of the following markers; CD27, HLA-F, HLA-B, HLA-A, B2M, HLA-E, Act CD4, Act CD8, Tem CD8, TAP2, HLA-DPB1, ICOS, TAP-1, HLA-C, HLA-DPA1, MDSC, ID01, CTLA-4, Treg, PD-L2, TIM3, PD-L1, TIGIT, PD-1, and LAG3.

44. (canceled)

45. The method of claim 23, wherein the method is performed prior to treatment of the patient with immunotherapy.

46. (canceled)

47. (canceled)

48. The method of claim 23, wherein the prostate cancer is biochemically recurrent prostate cancer, metastatic prostate cancer, and/or metastatic castrate-resistant prostate cancer.

49. The method of claim 23, wherein the method is performed after the patient undergoes radical prostatectomy.

50. The method of claim 23, further comprising performing a radical prostatectomy on the patient.

51. (canceled)

52. (canceled)

53. The method of claim 23, wherein the biological sample is a biopsy.

54. The method of claim 23, wherein the biological sample is a tumor sample.

55. The method of claim 23, wherein the patient is black or of African descent.

56. (canceled)

57. (canceled)

58. The method of claim 23, wherein the sample comprises prostate cancer cells and/or immune cells.

59. (canceled)

60. (canceled)
